(19) United States
(12) Patent Application Publication
(10) Pub. No.: US 2020/0102610 A1
(43) Pub. Date: Apr. 2, 2020

(54) METHOD FOR CEREBRAL PALSY PREDICTION

(71) Applicant: Bioscreening & Diagnostics LLC, Detroit, MI (US)

(72) Inventor: Ray Bahado-Singh, Grosse Pointe Shores, MI (US)

(73) Assignee: Bioscreening & Diagnostics LLC, Detroit, MI (US)

(21) Appl. No.: 16/589,307

(22) Filed: Oct. 1, 2019

Related U.S. Application Data
(63) Continuation of application No. 62/739,597, filed on Oct. 1, 2018.

(51) Int. Cl. C12Q 1/6869

(52) U.S. Cl. CPC C12Q 1/6869; C12Q 2600/112; C12Q 2600/154

(57) ABSTRACT

The present disclosure describes significant differences in methylation of cytosine bases in many loci throughout the genome in cases of cerebral palsy (CP) compared to unaffected cases (without CP). The present disclosure also describes novel methods for the prediction of CP that can be applied to embryos, fetuses, newborns, and different stages of postnatal life including childhood and any time in later postnatal life. The method is applicable to deoxyribonucleic acid (DNA) found in body fluids of CP subjects. Statistical techniques for estimating a subject's risk of having CP include comparing the degree of methylation of specific cytosine loci throughout the DNA in a subject being tested and comparing this to the percentage of cytosine at said sites in populations of individuals: with CP and/or a reference population of normal cases without CP. Risk for having specific types of CP or CP overall can also be determined based.

### CROSS REFERENCE TO RELATED APPLICATION

This application claims the benefit of U.S. Provisional Application No. 62/739,597 filed Oct. 1, 2018, which incorporated herein by reference in its entirety.

### FIELD

The present disclosure describes methods for predicting, detecting, and/or diagnosing cerebral palsy (CP).

### BACKGROUND

An international workshop (sponsored by the United Cerebral Palsy Research and Educational Foundation in Washington and the Castang Foundation in the UK) on definition and classification of Cerebral Palsy, held in Bethesda, Maryland in 2004, defined CP as follows:
Cerebral palsy (CP) describes a group of disorders of the development of movement and posture, causing activity limitation, that are attributed to non-progressive disturbances that occurred in the developing fetal or infant brain. The motor disorders of cerebral palsy are often accompanied by disturbances of sensation, cognition, communication, perception, and/or behavior, and/or by a seizure disorder.¹
   In 2006, an updated document on definition and classification of CP was offered for international consensus and adoption.²

Cerebral palsy (CP) is the most common motor disability in childhood that affects a person's ability to move and maintain balance and posture. Cerebral white matter lesions result in impaired motor development, motor control, muscle tone irregularities and abnormal reflexes and reactions.³ CP is one of a large heterogeneous group of neurodevelopmental, movement and posture disorders.^{4,5} Brain injury causes CP before, during, or after birth. Other associated impairments include attention deficit, cognition, perception, vision abnormalities, epilepsy, and intellectual abilities.^{6,7} Cerebral Palsy is more frequent in males than females⁸ and also more common among black children than white children.⁹

The estimated prevalence of CP in the United States population is 3 to 4 cases per 1000 live births.¹⁰ Most of the children identified with CP have spastic CP.¹¹ Many of the children with CP have at least one co-occurring condition including 30-50% cases with epilepsyl¹² and 7% with co-occurring Autism Spectrum Disorders (ASD).¹³ The prevalence of ASD among children with CP is much higher than among their peers without CP.

Cerebral Palsy can be caused by both genetic and environmental factors. A few of the major environmental trigger factors leading to CP include viral and bacterial intrauterine infections, intrauterine growth restrictions, antepartum hemorrhage, oxygen deprivation, complex pregnancies, preterm birth, low birth weight, placental complications, fetal strokes, bleeding in the brain, trauma to the developing fetus and exposure to toxins during critical stages of development.¹⁴

Despite the importance of CP, there is no single laboratory test for the routine population screening of embryos, fetuses, newborns or in later stages of post-natal life for CP. There is a significant need for screening tests that will facilitate the early identification of, medical surveillance of, and early treatment of newborns and other individuals at risk-for or with CP.

### SUMMARY

This Summary is not intended to identify key features or essential features of the claimed subject matter, nor is it intended to be used to limit the scope of the claimed subject matter.

The present disclosure describes identification and quantification of differences in the chemical structure of the cytosine nucleotide component of the DNA, so-called DNA methylation, in newborns and other individuals with cerebral palsy (“CP”) compared to normal (“unaffected”, “control”) cases i.e. without CP, for the purpose of determining the risk or likelihood of a tested individual having CP. Because of the universal presence of DNA in human cells and tissues, and also DNA released from dead cells, i.e., outside of cells but present on body fluids, the technique is applicable to any of these sources of DNA during the prenatal period and any time after birth, for the purposes of estimating risk or likelihood of an individual having CP. As noted, the disclosure also applies to DNA that has been released from cells that have undergone destruction, so-called cell-free DNA (cfDNA), and which is found in multiple different body fluids of individuals.

The chemical changes described, so-called “DNA methylation,” involve the addition of an extra carbon atom (—C—) to the cytosine component nucleotide, one of the known building blocks of DNA. Comparison of differences in cytosine nucleotide methylation at multiple loci or sites throughout the DNA is compared between CP and non-CP control groups or populations. When CpG methylation levels of an individual undergoing testing is compared to corresponding loci in these two reference population groups, the likelihood of CP can be determined. Any source of DNA from any tissue can be used for the methylation studies to predict CP risk at any stage of prenatal or postnatal life provided the appropriate reference populations are used.

### BRIEF DESCRIPTION OF THE DRAWINGS

FIG. 1. Receiver operating characteristic (ROC) curve analysis of methylation summaries for four specific markers linked with CP. The study identified 220 differentially-methylated CpG sites in 262 genes that each have an area under the ROC curve≥0.75 (p-val ≥0.05) for CP prediction. (chr 13; cg01561596; UFM1) (chr 3; cg03586379; SLC25A36) (chr 9; cg08052428; RALGDS) (chr 1; cg07898899; S100A13). AUC: Area Under the Receiver Operating Characteristics Curve; 95% CI: 95% Confidence Interval. Lower and upper Confidence Intervals are given in parentheses.

FIG. 2. Ingenuity pathway analysis (IPA) results for 262 gene Pathways included in the analysis. These genes were the most highly differentially methylated in association with CP. IPA results indicated the differentially methylated genes and gene networks are plausibly related to CP development, including: neuromotor damage, malformation of major brain structures, brain growth, neuroprotection, neuronal development and dedifferentiation, and cranial sensory neuron development.

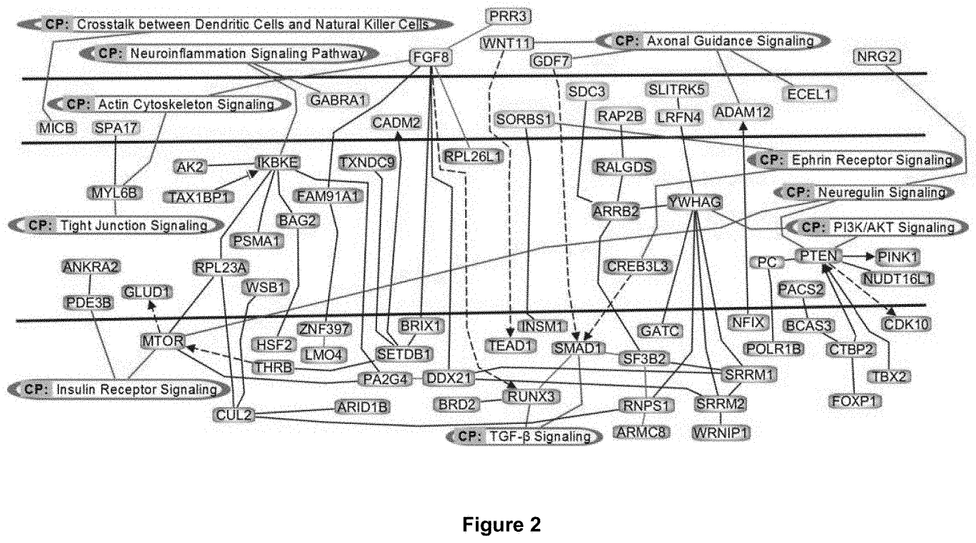

FIG. 3A. Hierarchical clustering segregated the samples into four distinct clusters comprising CP and normal controls. Heatmap of highly differentially methylated loci. Most highly differentially methylated loci represent the (False Detection Rate<0.000001). These CpG targets were with either 2.0-fold change in methylation and 10% methylation variation in the CP compared to normal patients. Direction, probe relationship and probe annotation, Fold change, differentially methylated CpG sites are also displayed. The top 25 CpG sites provided good discrimination of the CP cases from the controls as shown in the Heat Map.

FIG. 3B. Principal component analysis (PCA). Good segregation or clustering of CP cases from controls were achieved using 3 principal components (features or predictive markers). The percentages on the axes indicate the percentage contribution of each principal component (e.g. PC1) to our ability to segregate or separate the CP cases from controls.

### DETAILED DESCRIPTION

Cerebral palsy (CP) is a disorder of movement and posture that results from a non-progressive disorder of brain development. It is diagnosed clinically and has multiple etiological pathways: antenatal, perinatal, neonatal and post neonatal in timing of onset. The prevalence of CP in US and the world has remained stable over the past 40 years. The most common type of CP is spastic. Preterm babies are at increased risk for CP but more than 50% of children diagnosed with CP are born at term. Neonatal risk factors have been shown to have the greatest association with CP. Neuroimaging patterns show white matter injury as the most frequent. The clustering of CP in groups with high consanguinity and increased familial risk for CP suggests a genetic contribution. Despite the reported associations of several Single Nucleotide Polymorphisms (SNPs) for CP, results still remain controversial. Putative mechanisms for CP, including prenatal asphyxia, periventricular leukomalacia and hypoxic ischemic encephalopathy, are known to cause epigenetic modification of the genes.

There are four major types of CP: spastic, dyskinetic, ataxic, and mixed CP. Patients with spastic CP have increase muscle tone, which means their muscles are stiff and therefore, their movements are awkward. Patients with dyskinetic CP have problems controlling the movement of their hands, feet, and legs, so their movements can be slow or rapid and jerky. Sometimes, the face and tongue are also affected, and the patient has difficulty swallowing and talking. Patients with ataxic CP have poor balance and coordination, e.g. unsteady gait or have difficulty controlling hand movement when reaching to grasp or during writing. Patients with mixed CP have symptoms of more than one type of CP. An example of mixed CP is spastic-dyskinetic CP. Of the different types of CP, the spastic type is the most common.

Numerous studies have used different approaches in an attempt to find genetic associations with CP, including a Single Nucleotide Polymorphism (SNP) association study, haplotype analysis, linkage study, Copy Number Variation study, and whole exome and whole genome sequencing. These studies have identified number of genes and their sequence variations associated with clinical CP. One such study proposed that dysregulation of methylation capacity and folate one-carbon metabolism is causal for CP. Taken together, these studies support the conclusion that CP is associated with complex genetic factors.

The increased frequency of CP in groups with high rates of consanguinity, and observations of increased familial risk for CP further suggests a genetic contribution to CP. Accumulating evidence supports the theory that multiple genetic factors contribute to the cause of cerebral palsy. Mutations in multiple genes result in mendelian disorders that present with cerebral palsy-like features, and several single-gene mutations have been identified in idiopathic cerebral palsy pedigrees. Higher concordance rate for cerebral palsy in monozygotic twins than in dizygotic twin pair and also the effect of paternal age in some forms of cerebral palsy, further supports the theories of genetic alterations in CP.

Several genetic polymorphisms have been associated with susceptibility for CP, including apolipoprotein E, thrombophilia genes, and inflammation genes such as cytokines.

The term “epigenetics” represents the interaction between genes and the environment. These interactions do not result in changes to the genome itself yet contribute to variations in phenotypic expression. Epigenetic modifications are a major mechanism by which injury and destructive prenatal environmental factors can lead to long-term disturbances of brain development. During the acute and secondary phases of brain injury there is substantial loss of histone acetylation and methylation tags and considerable variation in microRNA expression. Reduced acetylation is associated with cognitive decline, which is accelerated after brain injury. Changes to epigenetic processes might be particularly relevant for white matter consistent with a recently established a model of white matter injury in which chronic perinatal inflammation, was induced by IL-1B exposure for the first 5 days after birth. As noted previously, epigenetic dysregulation occurs in important risk factors for CP, such as perinatal asphyxia, periventricular leukomalacia and hypoxic ischemic encephalopathy, and provides putative evidence for a role of epigenetic changes in CP development.

### Screening and Treatment Interventions for Cerebral Palsy

Screening for CP. CP is typically diagnosed between 12-24 months of age. A series of neurological tests, are generally used in different high-risk groups to monitor for CP development in at-risk groups. These include Dubowitz tests for newborns, the Hammersmith infant neurological examination (HINE) test, a modification of the Dubowitz test for older infants, Prechtl evaluation used in newborns, Touwen infant neurological exam (TINE), and the Ameil-Tison neurological evaluation test are available as briefly reviewed elsewhere. These reportedly have a sensitivity and specificity ranging from 88-92%

The General Movement Assessment (GMA) is the most widely used such test. Movement assessment is believed to reflect the intactness of neuronal circuitry in the brain including in the white matter. Serial assessment using GMA up to age 3-4 months is said to have sensitivity of 50-100% (median 98%) and specificity range of 35-100% (median 94%) suggesting significant variability.

Neuroimaging techniques are also widely used. Meta-analysis indicates that cranial ultrasound in premature newborns has an approximate 74% sensitivity and 92% specificity for predicting CP in high-risk individuals. MRI has good predictive accuracy for CP. A sensitivity of 86% and specificity of 89% has been reported for term MRI for predicting CP development by 31 months of age. MRI has significant limitations however including the high cost and time-consuming nature, and high level of professional expertise required to interpret the results, effectively disqualifying MRI as a screening tool.

Early treatment interventions for CP. There is evidence that early intervention can be beneficial in children with CP at least in the short term. Meta-analysis data indicated that general developmental programs does improve cognitive development up until age 3 years old. The infant health and development program (IHDP) approach was used in infants with low birth weight and reportedly ultimately resulted in improved performance in tests of vocabulary and mathematical abilities in babies with birthweight of 2000-2500 grams. The above interventions refer to high at-risk groups that do not necessarily end up with a diagnosis of CP.

The American Academy of Pediatrics (AAP) has however outlined the benefits of early diagnosis. This includes the opportunity for early, timely intervention at critical times of brain development, and improved motor and cognitive improvements when therapy is started as early as possible. In addition, the AAP emphasizes the significant family benefits to early CP diagnosis including allowing families earlier access to medical, psychosocial and financial resources provided by insurance and government agencies.

A clear advantage of the method described herein is that it is an epigenetic approach that permits prediction, detecting and/or diagnosis of CP in newborns, allowing early surveillance, diagnosis, intervention and improve CP outcomes and family well-being -as advocated by AAP. Such detection and/or diagnosis can be accomplished or facilitated in the neonatal period significantly earlier than the 12-24 months average gestational age at which CP is currently diagnosed. Predicting involves predicting the risk of the subjects of having CP. The present disclosure also describes a method for predicting the risk of subjects of having CP.

The present disclosure confirms highly significant differences in the percentage methylation of cytosine nucleotides throughout the genome in individuals with common categories of CP and normal groups using a widely available commercial bisulfite-based assay for distinguishing methylated from unmethylated cytosine. What is unique about the method described herein is that cytosines analyzed were not limited to CpG islands or to specific genes but included cytosine loci outside of CpG islands and outside of genes. For the purposes of this particular disclosure, cytosine loci associated with known genes and cytosines outside of known genes whose relationship to particular genes may be unknown were reported. The data provided in the Examples show significant differences in cytosine methylation loci throughout the genome between CP and unaffected controls. Likewise, cytosine methylation differences between individual CP-subcategories and each other and between individual CP subcategories and unaffected controls are identifiable and usable for the determining the different types of CP. The combination can be used as a lab test for the detection of or prediction of CP to further improve CP detection.

The term “control” refers to subjects that are normal or do not have CP. In embodiments, the control includes one or more normal subjects or subjects that do not have CP. The control is a well characterized population of one or more normal subjects or subjects that do not have CP. In embodiments, the cytosine methylation level of the patient being diagnosed is compared to that of a control.

In embodiments, the cytosine methylation level of the patient can also be compared to that of a CP patient group. CP patient group refers to one or more patients known to have CP, for example a well characterized population of one or more patients known to have CP. In embodiments, the cytosine methylation level of the patient being diagnosed is compared to that of a control and/or of a CP patient group.

Particular aspects provide panels of known and identifiable cytosine loci throughout the genome whose methylation levels (expressed as percentages) is useful for distinguishing CP from normal cases.

Additional aspects describe the capability of combining other recognized CP risk factors including but not limited to gestational age at delivery/ prematurity, inflammation/infection, placental histological abnormality, ultrasound or MRI brain findings, family history, maternal exposure to various toxins such as alcohol and tobacco (during the relevant pregnancy) along with cytosine methylation data for the prediction of CP. Multiple individual cytosine loci demonstrate highly significant differences in the degree of their methylation in CP versus control cases (FDR q-values 1.0×10⁻³ to 1.0×10⁻³⁵) see below.

Cytosine refers to one of a group of four building blocks “nucleotides” from which DNA is constructed. The other nucleotides or building blocks found in DNA are thiamine, adenine, and guanosine. The chemical structure of cytosine is in the form of a six-sided hexagon or pyrimidine ring.

The term methylation refers to the enzymatic addition of a “methyl group” or single carbon atom to position #5 of the pyrimidine ring of cytosine which leads to the conversion of cytosine to 5-methyl-cytosine. The methylation of cytosine as described is accomplished by the actions of a family of enzymes named DNA methyltransferases (DNMT's). The 5-methyl-cytosine when formed is prone to mutation or the chemical transformation of the original cytosine to form thymine. 5-methyl-cytosines account for about 1% of the nucleotide bases overall in the normal genome.

The term hypermethylation refers to increased frequency or percentage methylation at a particular cytosine locus when specimens from an individual or group of interest is compared to a normal or control group.

Cytosine is usually paired with guanosine another nucleotide in a linear sequence along the single DNA strand to form CpG pairs. “CpG” refers to a cytosine-phosphate-guanosine chemical bond in which the phosphate binds the two nucleotides together. In mammals, in approximately 70-80% of these CpG pairs the cytosine is methylated. The term “CpG island” refers to regions in the genome with high concentration of CG dinucleotide pairs or CpG sites. “CpG islands” are often found close to genes in mammalian DNA. The length of DNA occupied by the CpG island is usually 300-3000 base pairs. The CG cluster is on the same single strand of DNA. The CpG island is defined by various criteria including that the length of recurrent CG dinucleotide pairs occupying at least 200 bp of DNA and with a CG content of the segment of at least 50% along with the fact that the observed/expected CpG ratio should be greater than 60%. In humans about 70% of the promoter regions of genes have high CG content. The CG dinucleotide pairs may exist elsewhere in the gene or outside of and not know to be associated with a particular gene.

Approximately 40% of the promoter region (region of the gene which controls its transcription or activation)³⁶ of mammalian genes have associated CpG islands and three quarters of these promoter-regions have high CpG concentrations. Overall in most CpG sites scattered throughout the DNA the cytosine nucleotide is methylated. In contrast in the, CpG sites located in the CpG islands of promoter regions of genes the cytosine is unmethylated suggesting a role of methylation status of cytosine in CpG Islands in gene transcriptional activity.

The methylation of cytosines associated with or located in a gene is classically associated with suppression of gene transcription. In some genes however, increased methylation has the opposite effect and results in activation or increased transcription of a gene. One potential mechanism explaining the latter phenomenon could be through the inhibition of gene suppressor elements thus releasing the gene from inhibition. Epigenetic modification, including DNA methylation, is the mechanism by which for example cells which contain identical DNA are able to activate different genes and result in the differentiation into unique tissues e.g. heart or intestines.

Epigenetics is defined as heritable (i.e. passed onto offspring) changes in gene expression of cells that are not primarily due to mutations or changes in the sequence of nucleotides (adenine, thiamine, guanine, and cytosine) in the genes. Rather, epigenetics is a reversible regulation of gene expression by several potential mechanisms. One such mechanism which is the most extensively studied is DNA methylation. Other mechanisms include changes in the 3-dimensional structure of the DNA, histone protein modification, and micro-RNA inhibitory activity.

The receiver operating characteristics (ROC) curve is a graph plotting sensitivity-defined in this setting as the percentage of CP cases with a positive test or abnormal cytosine methylation levels at a particular cytosine locus on the Y axis and false positive rate (1-specificity)—i.e. the number of normal non-CP cases with abnormal cytosine methylation at the same locus—on the X-axis. Specificity is defined as the percentage of normal cases with normal methylation levels at the locus of interest or a negative test. False positive rate refers to the percentage of normal individuals falsely found to have a positive test (i.e. abnormal methylation levels).

The area under the ROC curves (AUC) indicates the accuracy of the test in identifying normal from abnormal cases.

The AUC is the area under the ROC plot from the curve to the diagonal line from the point of intersection of the X- and Y- axes and with an angle of incline of 45°. The higher the area under receiver operating characteristics (ROC) curve the greater is the accuracy of the test in predicting, diagnosing, or detecting the condition of interest. An area ROC=1.0 indicates a perfect test, which is positive (abnormal) in all cases with the disorder and negative in all normal cases (without the disorder). Methylation assay refers to an assay, a large number of which are commercially available, for distinguishing methylated versus unmethylated cytosine loci in the DNA.

Methylation Assays. Several quantitative methylation assays are available. These include COBRA™ which uses methylation sensitive restriction endonuclease, gel electrophoresis and detection based on labeled hybridization probes. Another available technique is the Methylation Specific PCR (MSP) for amplification of DNA segments of interest. This is performed after sodium ‘bisulfite’ conversion of cytosine using methylation sensitive probes. MethyLight™, a quantitative methylation assay-based uses fluorescence-based PCR. Another method used is the Quantitative Methylation (QM™) assay, which combines PCR amplification with fluorescent probes designed to bind to putative methylation sites. Ms-SNuPE™ is a quantitative technique for determining differences in methylation levels in CpG sites. As with other techniques bisulfite treatment is first performed leading to the conversion of unmethylated cytosine to uracil while methyl cytosine is unaffected. PCR primers specific for bisulfite converted DNA is used to amplify the target sequence of interest. The amplified PCR product is isolated and used to quantitate the methylation status of the CpG site of interest. The preferred method of measurement of cytosine methylation is the Illumina method. Whole genome methylation sequencing to identify methylation levels of each CpG loci throughout the genome and whole exome sequencing to identify the level of methylation for each CpG loci throughout the exomes may also be performed to determine methylation differences between CP cases and unaffected controls.

IIlumina Method. For DNA methylation assay the Illumina Infinium® Human Methylation 450 Beadchip assay was used for genome wide quantitative methylation profiling. Briefly genomic DNA is extracted from cells in this case archived blood spot, for which the original source of the DNA is white blood cells. Using techniques widely known in the trade, the genomic DNA is isolated using commercial kits. Proteins and other contaminants were removed from the DNA using proteinase K. The DNA is removed from the solution using available methods such as organic extraction, salting out or binding the DNA to a solid phase support. Bisulfite Conversion

Bisulfite Conversion. As described in the Infinium® Assay Methylation Protocol Guide, DNA is treated with sodium bisulfite which converts unmethylated cytosine to uracil, while the methylated cytosine remains unchanged. The bisulfite converted DNA is then denatured and neutralized. The denatured DNA is then amplified. The whole genome application process increases the amount of DNA by up to several thousand-fold. The next step uses enzymatic means to fragment the DNA. The fragmented DNA is next precipitated using isopropanol and separated by centrifugation. The separated DNA is next suspended in a hybridization buffer. The fragmented DNA is then hybridized to beads that have been covalently limited to 50 mer nucleotide segments at a locus specific to the cytosine nucleotide of interest in the genome. There is a total of over 500,000 bead types specifically designed to anneal to the locus where the particular cytosine is located. The beads are bound to silicon-based arrays. There are two bead types designed for each locus, one bead type represents a probe that is designed to match to the methylated locus at which the cytosine nucleotide will remain unchanged. The other bead type corresponds to an initially unmethylated cytosine which after bisulfite treatment is converted to a thiamine nucleotide. Unhybridized (not annealed to the beads) DNA is washed away leaving only DNA segments bound to the appropriate bead and containing the cytosine of interest. The bead bound oligomer, after annealing to the corresponding patient DNA sequence, then undergoes single base extension with fluorescently labeled nucleotide using the ‘overhang’ beyond the cytosine of interest in the patient DNA sequence as the template for extension.

If the cytosine of interest is unmethylated then it will match perfectly with the unmethylated or “U” bead probe. This enables single base extensions with fluorescent labeled nucleotide probes and generate fluorescent signals for that bead probe that can be read in an automated fashion. If the cytosine is methylated, single base mismatch will occur with the “U” bead probe oligomer. No further nucleotide extension on the bead oligomer occurs however thus preventing incorporation of the fluorescent tagged nucleotides on the bead. This will lead to low fluorescent signal form the bead “U” bead. The reverse will happen on the “M” or methylated bead probe.

Laser is used to stimulate the fluorophore bound to the single base used for the sequence extension. The level of methylation at each cytosine locus is determined by the intensity of the fluorescence from the methylated compared to the unmethylated bead. Cytosine methylation level is expressed as “β” which is the ratio of the methylated bead probe signal to total signal intensity at that cytosine locus. These techniques for determine cytosine methylation have been previously described and are widely available for commercial use.

The current disclosure describes the use of a commercially available methylation technique to cover up to 99% Ref Seq genes involving approximately 16,000 genes and 500,000 cytosine nucleotides down to the single nucleotide level, throughout the genome (Infinium Human Methylation 450 Beach Chip Kit). The frequency of cytosine methylation at single nucleotides in a group of CP cases compared to controls is used to estimate the risk or probability of CP. The cytosine nucleotides analyzed using this technique included cytosines within CpG islands and those at further distances outside of the CpG islands i.e. located in “CpG shores” and “CpG shelves” and even more distantly located from the island so called “ CpG seas”.

Identification of Specific Cytosine Nucleotides. Reliable identification of specific cytosine loci distributed throughout the genome has been detailed (Illumnia) in the document: “CpG Loci Identification. A guide to Illumina's method for unambiguous CpG loci identification and tracking for the GoldenGate® and Infinium™ assays for Methylation”. A brief summary follows. Illumina has developed a unique CpG locus identifier that designates cytosine loci based on the actual or contextual sequence of nucleotides in which the cytosine is located. It uses a similar strategy as used by NCBI's re SNP IPS (rs#) and is based on the sequence flanking the cytosine of interest. Thus, a unique CpG locus cluster ID number is assigned to each of the cytosine undergoing evaluation. The system is reported to be consistent and will not be affected by changes in public databases and genome assemblies. Flanking sequences of 60 bases 5′ and 3′ to the CG locus (i.e. a total of 122 base sequences) is used to identify the locus. Thus, a unique “CpG cluster number” or cg# is assigned to the sequence of 122 bp which contains the CpG of interest. The cg# is based on Build 37 of the human genome (NCBI37). Accordingly, only if the 122 bp in the CpG cluster is identical, there is a risk of a locus being assigned the same number and being located in more than one position in the genome. Three separate criteria are utilized to track individual CpG locus based on this unique ID system. Chromosome number, genomic coordinate and genome build. The lesser of the two coordinates “C” or “G” in CpG is used in the unique CG loci identification. The CG locus is also designated in relation to the first ‘unambiguous” pair of nucleotides containing either an ‘A’ (adenine) to ‘T’ (thiamine). If one of these nucleotides is 5′ to the CG then the arrangement is designated TOP and if such a nucleotide is 3′ it is designate BOT.

In addition, the forward or reverse DNA strand is indicated as being the location of the cytosine being evaluated. The assumption is made that methylation status of cytosine bases within the specific chromosome region is synchronized.

Description of the Method. A single neonatal dried blood spot saved on filter paper was retrieved from biobank specimens collected as part of the well-established Michigan newborn screening program for the detection of metabolic disorders and stored by the Michigan Department of Community Health (MDCH) in Lansing, Mich. Blood was originally obtained by heel-stick and placed on filter paper generally an average of 2 days after birth. Samples were stored at room temperature. De-identified residual blood spots after the completion of clinical testing were used. IRB approval was obtained by a standardized process through the MDCH. The specimens used for the current study were collected between 1998 and 2003. Cases with chromosomal abnormalities or other known or suspected genetic syndromes or the presence of accompanying major birth defects were excluded.

A total of 23 cases of CP, along with a total of 21 controls were analyzed. Control cases were neurologically normal children at the time of chart review and at patient reporting and with no known or suspected birth defects or genetic syndromes. CP as a single group was compared to unaffected controls.

In embodiments, the present disclosure describes a method for predicting, diagnosing, and/or detecting CP based on measurement of frequency or percentage methylation of cytosine nucleotides in various identified loci in a DNA sample of a patient in need thereof. The method includes obtaining a sample from a patient; extracting DNA from the sample; assaying the sample to determine the percentage methylation of cytosine at loci throughout genome; comparing the cytosine methylation level of the patient to a control; and calculating the individual risk of CP based on the cytosine methylation level at different CpG sites throughout the genome. In embodiments, the patient could be an embryo, a fetus, a new born, or a pediatric patient in need of determining whether the patient has CP. DNA used can originate from any cell or tissue or body fluid which need not be limited to blood. DNA can be obtained from maternal body fluid, such as maternal blood. For example, DNA obtained from buccal swab is one source that could be used. The control could be a well characterized group of normal (healthy) or more precisely individuals unaffected by neurologic disorders, people matched against a well characterized population of CP patients. The well characterized group of normal people or CP patients may include one or more normal people or CP patients or may include a population of normal people or CP patients. The control group of normal people or CP patients could be fetus, embryo, a newborn, or a pediatric patient.

The present method provides predicting, detection, and/or diagnosis of patients with CP. The present method also provides early prediction, detection and/or diagnosis of CP. In embodiments, the patient is an embryo or fetus. The DNA of the fetus or embryo can be obtained from maternal blood. Early prediction, detection, and/or diagnosis of CP include prediction, detection, and/or diagnosis of CP while the patient is a fetus or an embryo, before the patient is born. In embodiments, the prediction of CP includes predicting the risk of the patient having CP.

DNA Extraction from Blood-Spot. DNA extraction was performed as described in the EZ1® DNA Investigator Handbook, Sample and Assay Technologies, QIAGEN 4^{th} Edition, April 2009. A brief summary of the DNA extraction method is provided. Two 6 mm diameter circles (or four 3 mm diameter circles) were punched out of a dried blood spot stored on filter paper and used for DNA extraction. The circle contains DNA from white blood cells from approximately 5 μL of whole blood. The circles are transferred to a 2 ml sample tube.

A total of 190 μL of diluted buffer G2 (G2 buffer: distilled water in 1:1 ratio) was used to elute DNA from the filter paper. Additional buffer was added until residual sample volume in the tube is 190 μL since filter paper absorbs a certain volume of the buffer. Ten μL of proteinase K is added and the mixture is vortexed for 10 s and quick spun. The mixture is then incubated at 56° C. for 15 minutes at 900 rpm. Further incubation at 95° C. for 5 minutes at 900 rpm is performed to increase the yield of DNA from the filter paper. Quick spin was performed. The sample is then run on EZ1 Advanced (Trace, Tip-Dance) protocol as described. The protocol is designed for isolation of total DNA from the mixture. Elution tubes containing purified DNA in 50 μL of water is now available for further analysis.

Infinium DNA Methylation Assay. Methylation Analysis-Illumina's Infinium Human Methylation 450 Bead Chip system was used for genome-wide methylation analysis. DNA (500 ng) was subjected to bisulfite conversion to deaminate unmethylated cytosines to uracils with the EZ-96 Methylation Kit (Zymo Research) using the standard protocol for Infinium. The DNA is enzymatically fragmented and hybridized to the Illumina BeadChips. BeadChips contain locus-specific oligomers and are in pairs, one specific for the methylated cytosine locus and the other for the unmethylated locus. A single base extension is performed to incorporate a biotin-labeled ddNTP. After fluorescent staining and washing, the BeadChip is scanned and the methylation status of each locus is determined using BeadStudio software (Illumina). Experimental quality was assessed using the Controls Dashboard that has sample-dependent and sample-independent controls target removal, staining, hybridization, extension, bisulfite conversion, specificity, negative control, and non-polymorphic control. The methylation status is the ratio of the methylated probe signal relative to the sum of methylated and unmethylated probes. The resulting ratio indicates whether a locus is unmethylated (0) or fully methylated (1). Differentially methylated sites are determined using the Illumina Custom Model and filtered according to p-value using 0.05 as a cutoff.

IIlumina's Infinium HumanMethylation450 BeadChip system, an updated assay method that covers CpG sites (containing cytosine) in the promoter region of more genes, i.e., approximately ˜16,880. In addition other cytosine loci throughout the genome and outside of genes, and within or outside of CpG islands are represented in this assay.

Validation by pyrosequencing. It was confirmed that the methylation state inferred by the Illumina HumanMethylation450K arrays data was not biased, but represented true changes. The top 25 genes were selected for independent validation by pyrosequencing, based on their % methylation, AUC ROC, top fold change and EDR p-values. These analyses revealed similar methylation data as those calculated from the Illumina HumanMethylation450K arrays for all 25 genes. We examined bisulfite-converted genomic DNA by quantitative pyrosequencing analysis. Detailed methodology was published previously.

Cytosine Methylation for the Prediction of CP Risk Using ROC Curve. To determine the accuracy of the methylation level of a particular cytosine locus for CP prediction, different threshold levels of methylation e.g. ≥10%, ≥20%, ≥30%, ≥40% etc. at the site was used to calculate sensitivity and specificity for CP prediction. Thus, for example using ≥10% methylation at a particular cg locus, cases with methylation levels above this threshold would be considered to have a positive test and those with lower than this threshold are interpreted as a negative methylation test. The percentage of CP cases with a positive test in this example 10% methylation at this particular cytosine locus would be equal to the sensitivity of the test. The percentage of normal non-CP cases with cytosine methylation levels of <10% at this locus would be considered the specificity of the test. False positive rate is here defined as the percentage of normal cases with a (falsely) abnormal test result and sensitivity is defined as the pecentage of CP cases with (correctly) abnormal test result i.e. the level of methylation ≥10% at this particular cg location. A series of threshold methylation values are evaluated e.g. ≥ 1/10, ≥ 1/20, ≥ 1/30 etc., and used to generate a series of paired sensitivity and false positive values for each locus. A receiver operating characteristic (ROC) curve which is a plot of data points with sensitivity values on the Y-axis and false positivity rate (1-specificity) on the X-axis is generated. This approach can be used to generate ROC curves for each individual cytosine locus that displays significant methylation differences between cases and CP groups. The computer program “R” (version 3.2.2.) was used to calculate the AUC and 96% CI's.

Standard statistical testing using p-values to express the probability that the observed difference between cytosine methylation at a given locus between CP and control DNA specimens were performed.

More stringent testing using False Discovery Rate (FDR) was also performed. The FDR gives the probability that positive results were due to chance when multiple hypothesis testing is performed using multiple comparisons.

In embodiments, using the Illumina Infinium Assays for whole genome methylation studies, significant differences in the frequency (level or percentage) of methylation of specific cytosine nucleotides associated with particular genes were demonstrated in the CP group individually when compared to a normal group. The differences in cytosine methylation levels are highly significant and of sufficient magnitude to accurately distinguish the CP from the normal group. Thus, the methods described herein can be used as a test to screen for CP cases among a mixed population with CP and normal cases.

The degree of methylation of cytosines could potentially vary based on individual factors (diet, race, age, gender, medications, toxins, environmental exposures, other concurrent medical disorders and so on). Overall, despite these potential sources of variability, whole genome cytosine methylation studies identified specific sites within (and outside of) certain genes and could distinguish and therefore could serve as a useful screening test for identification of groups of individuals predisposed to or at increased risk for having different categories of CP compared to normal cases.

Since cells, with few exceptions (mature red blood cells and mature platelets), contain nuclei and therefore DNA, the methods described herein can be used to screen for CP using DNA from any cells with the exception of the two named above. In addition, cell free DNA from cells that have been destroyed and which can be retrieved from body fluids can be used for such screening.

Cells and DNA from any biological samples which contain DNA can be used for the purpose of assessing or predicting CP in a patient. Assessing includes detecting and/or diagnosing. Samples used for testing can be obtained from living or dead tissue and also archeological specimens containing cells or tissues. Examples of biological specimens that can be used to obtain DNA for CP screening include: amniocytes, placental tissue, cell-free DNA in body fluids, skin, hair, follicles/roots, buccal and mucous membranes, internal body tissue, or placental or umbilical cord tissue obtained at birth. Examples of body fluids include blood, umbilical cord blood, saliva, genital or cervical secretions, urine, sweat, and tear. Examples of mucous membranes include cheek scrapings, buccal scrapings, or scrapings from the tongue.

DNA are obtained from biological samples of patients, such as from an embryo, a fetus, a new born, or a pediatric patient. When the patient is an embryo or fetus, the DNA can be obtained from a biological sample of the mother, the pregnant woman, carrying the embryo or fetus. The biological sample can be obtained from a pregnant woman in her first trimester, second trimester, or third trimester.

The biological sample can be a body fluid, such as blood, plasma, serum, urine, saliva, cervical secretion, and amniotic fluid. The biological sample can be tissue samples from the patient including placental tissue from a new born or of a fetus or embryo, blood from the mother or fetuses, amniocytes (fetal cells) from amniotic fluid. Amniocytes represent cells from fetal skin, respiratory tract, and gastrointestinal tract. The placental tissue can be obtained by placental biopsy or chorionic villus sampling (CVS). The biological sample can be placental tissue that is fresh or archived.

An “embryo” refers to the patient from the time of fertilization to the end of the eighth week of gestation. A “fetus” refers to the patient after the eighth week of gestation. When the patient is an embryo or a fetus, obtaining a biological sample from a patient includes obtaining a biological sample from the mother carrying the embryo or fetus. Accordingly, when the patient is an embryo or fetus, the mother can also be a patient.

Other embodiments include the use of genome-wide differences in cytosine methylation in DNA to screen for and determine risk or likelihood of CP at any stage of prenatal and postnatal life. These stages include the embryo, fetus, the neonatal period (first 28 days after birth), infancy (up to 1 year of age), childhood (up to 10 years of age, adolescence (11 to 21 years of age), and adulthood (i.e. >21 years of age).

The results presented herein confirm that based on the differences in the level of methylation of the cytosine sites between CP and normal cases throughout the whole human genome, the predisposition to or risk of having a CP overall or subcategories of CP can be determined.

The explanation for the differences in methylation is that the development of CP results from and/or is associated with changes induced by toxins, chemical agents, inflammation, oxygen deprivation, birth trauma, etc. that are known to be associated with causative risk factors and differing potency in CP development. Altered methylation leads to abnormal expression of multiple genes many of which directly or indirectly impact or control cardiac development. Abnormal gene function includes either the suppression of the function of genes whose activities are important to normal brain development or conversely the activation of genes whose functions are normally suppressed to permit normal development of the brain. Further, substances that affect the development of CP for example alcohol, could independently have an effect on other genes that have no relationship to brain development but based on “alcohol effect” develop methylation abnormalities. Thus, genome wide cytosine methylation study provides information on the orchestrated widespread activation and suppression of multiple genes and gene networks some of which are involved in the normal and abnormal development of the brain. The approach described herein does not require prior knowledge of the role of particular genes in brain development or the mechanism by which changes in the function of the genes lead to CP. Indeed, this approach can provide novel insights and explanations for mechanisms of CP development. Further, hundreds of thousands of cytosine loci involving thousands of genes are evaluated simultaneously and in an unbiased fashion and can thus be used to accurately estimate the risk of CP. Of further importance is the fact that cytosine loci outside of the genes can also control gene function, so methylation levels of loci situated outside of the gene further contribute to the prediction of CP.

In embodiments, the present disclosure confirms aberration or change in the methylation pattern of cytosine nucleotide occurs at multiple cytosine loci throughout the genome in individuals affected with different forms of CP compared to individuals with normal brain development.

In other embodiments, the present disclosure describes techniques and methods for predicting or estimating the risk of CP based on the differences in cytosine methylation at various DNA locations throughout the genome.

Currently no reliable clinically available biological method using cells, tissue or body fluids exist for predicting or estimating the risk of CP in individuals in the population.

CP overall was evaluated and compared to unaffected control groups and cytosine nucleotides displaying statistically significant differences in methylation status throughout the genome were identified. Because of the extended coverage of cytosine nucleotides, some differentially methylated cytosines were located outside of CpG islands and outside of known genes. DNA methylation changes in either intragenic or extragenic cytosines individually (or in any combinations) can be used to detect or predict the development of CP.

The present study reports a strong association between cytosine methylation status at a large number of cytosine sites throughout the genome using stringent False Discover Rate (FDR) analysis with q-values <0.05 and with many q-values as low as <1×10⁻³⁰, depending on particular cytosine locus being considered (Tables 1). A total of 23 cases of CP and 21 unaffected controls were evaluated. Significant differences in cytosine methylation patterns at multiple loci throughout the DNA that was found in all CP cases tested compared to normal. The particular cytosines disclosed are located in known genes. The findings are consistent with altered expression of multiple genes in CP cases compared to controls.

The cytosine methylation markers reported enables population screening studies for the prediction and detection of CP based on cytosine methylation throughout the genome. They also permit improved understanding of the mechanism of development of CP for example by evaluating the cytosine methylation data using gene ontology analysis.

The cytosine evaluated in the present application includes but are not limited to cytosines in CpG islands located in the promoter regions of the genes. Other areas targeted and measured include the so called CpG island ‘shores’ located up to 2000 base pairs distant from CpG islands and ‘shelves’ which is the designation for DNA regions flanking shores. Even more distant areas from the CpG islands so called “seas” were analyzed for cytosine methylation differences. The extragenic cytosine loci, located outside of known genes (however they could potentially maintain long-distance control of unspecified genes) also detected CP with moderate, good and excellent accuracy as indicated based on the AUROC. Thus, comprehensive and genome-wide analysis of cytosine methylation is performed.

Statistical Analyses. The present disclosure describes a method for estimating the individual risk of having CP or even a particular type of CP. This calculation can be based on logistic regression analysis leading to identification of the significant independent predictors among a number of possible predictors (e.g. methylation loci) known to be associated with increased risk of CP. Cytosine methylation levels at different loci can be used by themselves or in combination with other known risk predictors such as for example prenatal exposure to toxins -“yes” or “no” (e.g. gestational age at birth, maternal alcohol consumption, family history and methylation levels in a single or multiple loci) which are known to be associated with increased risk of the particular type of CP as described in this application. The probability of an affected individual can be derived from the probability equation based on the logistic regression:

P_{CP}=1/1+e−(^{B1x}₁+^{B2x}₂+^{B3x}₃ . . . ^{Bnx}ₙ)

where ‘x’ refers to the magnitude or quantity of the particular predictor (e.g. methylation level at a particular locus) and “β” or β- coefficient refers to the magnitude of change in the probability of the outcome (a particular type of CP) for each unit change in the level of the particular predictor (x) such as for example gender or gestational age (in weeks) at birth. The β values are derived from the results of the logistic regression analysis. “β-values” referred to herein are different than those obtained from Illumina. β-values in the laboratory analysis refers to the level/percentage of cytosine methylation. These statistically related β-values would however be derived from multivariable logistic regression analysis in a large population of affected and unaffected individuals. Values for ^{x,}₁ ^{,x}₂ ^{,x}₃ etc, representing in this instance methylation percentage at different cytosine locus would be derived from the individual being tested while the β-values would be derived from the logistic regression analysis of the large reference population of affected (CP) and unaffected cases mentioned above. Based on these values, an individual's probability of having a type of CP can be quantitatively estimated. Probability thresholds are used to define individuals at high risk (e.g. a probability of ≥1/100 of CP may be used to define a high risk individual triggering further evaluation such as neurological tests previously described, e.g. GMA or general movement assessment test, while individuals with risk <1/100 would require no further follow-up. The threshold used will among other factors be based on the diagnostic sensitivity (number of CP cases correctly identified), specificity (number of non-CP cases correctly identified as normal), and cost of other tests for CP. Logistic regression analysis is well known as a method in disease screening for estimating an individual's risk for having a disorder. Logistic regression analysis can be performed with established computer programs such as “R” program Logistic regression analysis can be performed with established computer programs such as “R” program (www.rprogramind.net) (version 3.2.2).

Specific Microarray Kits for Cerebral Palsy Detection. The present disclosure describes microarray chips developed for CP risk-estimation using DNA, including cf DNA, from various body tissues and body fluids. The Illumina HumanMethylation450 Array was primarily designed for such genomic analysis. Microarrays specific for genes involved in brain development and neurologic abnormalities can further improve predictive accuracy for CP detection. Such an approach could include but not be limited to more concentrated coverage of CpG loci (more CpG loci) within or associated with (extragenic) of genes identified herein as being differentially methylated and relevant brain, neuronal and neuromuscular genes. Assessing the methylation of multiple CpG loci that are close to a particular locus of interest (10-20 closest CpG loci in a given region rather than a single cpG locus) would allow average CpG methylation for that region to be calculated. An average methylation calculation would reduce chance variation in methylation levels due to experimental conditions and improve predictive accuracy.

An additional benefit of the method described herein is that the varied etiology and clinical presentation makes it very unlikely that single markers or single diagnostic technique can identify a high percentage of cases. The global approach represented by the whole genome epigenomics analysis greatly enhances the likelihood for accurate prediction of CP and its subgroups a leading to earlier diagnosis and therapeutic interventions as proposed by the AAP.

Individual risk of CP can also be calculated by using methylation percentages (reported as β-coefficients) at the individual discriminating cytosine locus by themselves or using different combinations of loci based on the method of overlapping Gaussian distribution or multivariate Gaussian distribution where the variable would be methylation level/percentage methylation at a particular (or multiple) loci so called. Alternatively, if methylation percentages or β-coefficients are not normally distributed (i.e. non-Gaussian), normal Gaussian distribution would be achieved if necessary by logarithmic transformation of these percentages.

As an example, two Gaussian distribution curves are derived for methylation at particular loci in the CP and the normal unaffected populations. Mean, standard deviation and the degree of overlap between the two curves are then calculated. The ratio of the heights of the distribution curves at a given level of methylation will give the likelihood ratio or factor by which the risk of having CP is increased (or decreased) at a particular level of methylation at a given locus. The likelihood ratio (LR) value can be multiplied by the background risk of CP (for a particular type of CP, or for CP overall) in the general population and thus give an individual's risk of CP based on methylation level at the cg site(s) chosen.

Differential methylation can be analyzed using a microarray system. Nucleic acids can be linked to chips, such as microarray chips. See, for example, U.S. Pat. Nos. 5,143,854; 6,087,112; 5,215,882; 5,707,807; 5,807,522; 5,958,342; 5,994,076; 6,004,755; 6,048,695; 6,060,240; 6,090,556; and 6,040,138. Binding to nucleic acids on microarrays can be detected by scanning the microarray with a variety of laser or charge coupled device (CCD)-based scanners, and extracting features with software packages, for example, Imagene (Biodiscovery, Hawthorne, Calif.), Feature Extraction Software (Agilent), Scanalyze (Eisen, M. 1999. SCANALYZE User Manual; Stanford Univ., Stanford, Calif. Ver 2.32.), or GenePix (Axon Instruments).

### Artificial Intelligence and Deep Learning Approaches

The present disclosure also describes the use of Artificial Intelligence and Deep Learning for detecting and/or diagnosing CP or predicting the risk of CP in subjects.

Deep Learning (DL). Generally classical machine learning techniques make predictions directly from a set of features that have been pre-specified by the user. However, representation learning techniques transform features into some intermediate representation prior to mapping them to final predictions. Deep Learning (DL) is a form of representation learning that uses multiple transformation steps to create very complex features. DL is widely applied in pattern recognition, image processing, computer vision, and recently in bioinformatics. DL is categorized into feed-forward artificial neural networks (ANNs), which uses more than one hidden layer (y) that connects the input (x) and output layer (z) via a weight (VV) matrix. The weight matrix W which is expected to minimize the difference between the input layer (x) and the output layer (z) is considered as the best one and chosen by the system to get the best results.

Machine Learning Algorithms (MLA). A representative set of five machine learning classification algorithms which have been applied for problems of data classification in metabolomics and genomics studies can be selected and the results of these five machine learning algorithms compared with deep learning. Random forest (RF) is a widely used machine learning algorithm based on decision tree theory. It works with high-dimensional data and can deal with unbalanced and missing values in the data. Support vector machine (SVM) is another machine learning algorithm that separates the metabolomics data with N data points into (N-1) dimensional hyperplane. SVM has the advantage of avoiding over-fitting and uses the kernel trick for more complex problems to get better results by changing the kernel function. Generalized Linear Model (GLM) measures the relationship between the categorical dependent variable and one or more independent variables by estimating probabilities using a logistic function, which is the cumulative logistic distribution. The output of a GLM is more informative than other classification algorithms. Prediction Analysis for Microarrays (PAM) is a statistical technique for class prediction from gene expression data using nearest shrunken centroids. This method identifies the subsets of genes that best characterize each class and gives satisfying results in metabolomics and genomics studies as well. Linear Discriminant Analysis (LDA) is closely related to analysis of variance (ANOVA) and regression analysis, which also attempt to express one dependent variable as a linear combination of other features or measurements.

Software Packages Utilized. The H2O R package (https://cran.r-project.org/web/packages/h2o/h2o.pdf, Author The H2O.ai team Maintainer Tom Kraljevic <tomk@0xdata.com>) was used to tune the parameters of the DL model.

To get the optimal predictions for the artificial intelligence algorithms other than DL, the caret R package (https://cran.r-project.org/web/packages/caret/caret.pdf, Maintainer Max Kuhn <mxkuhn@gmail.com>) was used to tune the parameters in the models.

The variable importance functions varimp in H2O and varImp in caret R packages were used to rank the models features in each of the predictive algorithms.

The pROC R package can be used to compute area under the curve (AUC) of a receiver-operating characteristic (ROC) curve to assess the overall performance of the models.

Modeling & Evaluation. The data can be split into 80% training set and 20% testing set. While dealing with a small and medium size of data in the machine learning applications, the 80/20 split is a commonly used one. A 10-fold cross validation was performed on the 80% training data during the model construction process, and the model was tested on the hold out 20% of data. To avoid sampling bias, the above splitting process was repeated ten times and calculated the average AUC on the 10 hold out test sets. In addition to AUC, sensitivity, specificity, and 95% confidence intervals for the test sets were calculated.

The following parameters can be used to tune the DL model and other machine learning algorithms: for DL model Epochs (number of passes of the full training set), I1 (penalty to converge the weights of the model to 0), I2 (penalty to prevent the enlargement of the weights), input dropout ratio (ratio of ignored neurons in the input layer during training), andnumber of hidden layers; for SVM model, cost of classification; for RF model, number of trees to fit; and for PAM model, threshold amount for shrinking toward the centroid.

To avoid overfitting in the DL model, three regularization parameters were used. L1, which increases model stability and causes many weights to become 0 and L2, which prevents weights enlargement. L1 lets only strong weights survive (constant pulling force towards zero), while L2 prevents any single weight from getting too big. Dropout has recently been introduced as a powerful generalization technique, and is available as a parameter per layer, including the input layer. The key idea is to randomly drop units (along with their connections) from the neural network during training. This prevents units from co-adapting too much. The third parameter used for avoiding overfitting in DL model is input_dropout_ratio which controls the amount of input layer neurons that are randomly dropped (set to zero), controls overfitting with respect to the input data (useful for high-dimensional noisy data).

Feature Importance. Feature (predictor) importance is estimated using a model-based approach. In other words, a feature is considered important if it contributes to the predictive model performance. Variable importance functions varimp in H2O and varImp in caret R packages were used to rank the models features in each of the predictive algorithms.

Using DL and machine learning (ML) techniques, the first data set, in this case 220 epigenomic biomarkers, can be divided up into 5 to 6 equal groups and analyzed separately. Each group can then be evaluated separately (epigenomic biomarker only) and also combined with the clinical and demographic predictors or risk factors for CP. Next, all the epigenomic biomarkers of the first data set in one group are analyzed to observe performance differences. The second data set or group of epigenetic markers as one group can then be analyzed to see the performance results of epigenomic markers with and without clinical and demographic markers. For every group, the top epigenomic markers or epigenomic and clinical markers are analyzed and ranked.

The aim is to assess the predictive ability of the DL framework to separate CP patients using genomics data. Toward this goal, preprocessing steps (log transformation, centering, autoscaling, and quantile normalization) are applied before constructing the DL model. Before training the model, the model is pre-trained using autoencoder on the whole data without labels. This step improves the model performance, avoids random initialization of the weights, and selects the best model architecture. Subsequently, the DL model is trained using a wide range of parameters (as stated in Modeling & Evaluation section) and selected the best model with the minimum mean square error.

DL is subsequently compared with five other commonly used artificial intelligence methods: RF, SVM, LDA, PAM, and GLM, bearing in mind the strengths of the different approaches. The average AUCs, sensitivity and specificity values calculated on the hold out (validation) test sets are then reported. Higher area under the ROC curve value is often achieved with DL than other AI methods. In addition, higher sensitivity and specificity values are often achieved with DL than other AI methods, too.

Diagnostic accuracy as represented by AUC (95% CI) was performed for individual CpG loci using the “R” computer program. The use of logistic regression analysis for calculation of overall diagnostic accuracy for CP detection using a combination of CpG loci can be performed using “R” logistic regression package (V3.2.2.). Logistic regression analysis can be used also for calculation of sensitivity and specificity for the prediction of CP based on methylation of cytosine loci.

It has been demonstrated that statistically highly significant differences exist in the percentage or level of methylation of individual cytosine nucleotides distributed throughout the genome both within and outside of the genes when cases with CP are compared to normal unaffected cases. Cytosines demonstrating methylation differences are distributed both inside and outside of (CpG islands, shores) and genes. The disclosure describes methylation markers for distinguishing individual categories of CP and CP overall from normal cases.

In embodiments, a panel of cytosine markers are described for distinguishing individual categories of CP from normal cases and also for distinguishing CP as a group from normal cases without CP. The disclosure includes risk assessment at any time or period during postnatal life.

In embodiments, measurements of cytosine methylation and its use in distinguishing common categories of CP from each other are described.

In embodiments, the use of statistical algorithms and methods for estimating the individual risk of CP based on methylation levels at informative cytosine loci are described.

In embodiments, methods for predicting, detecting, and/or diagnosing CP based on measurement of the frequency or percentage methylation of cytosine nucleotides in various identified loci in the DNA of subjects are described. The present disclosure describes a method comprising the steps of: A) obtaining a sample from a subject; B) extracting DNA from blood specimens; C) assaying to determine the percentage methylation of cytosine at loci throughout the genome; D) comparing the cytosine methylation level of the subject to a well characterized population of normal and CP groups; and E) calculating the individual risk of CP based on the cytosine methylation level at different sites throughout the genome.

The methods for predicting, detecting, and/or diagnosing CP described herein further includes using DL and ML for more accurately determining CP and/or estimating the risk of CP in a patient. In embodiments, methods described herein includes performing logistic regression. In embodiments, logistic regression includes using DL and MLA.

In embodiments, the sample from the patient is a biological sample which can be a tissue sample or a body fluid from the patient. Examples of body fluid includes blood, fetal blood umbilical cord blood, plasma, serum, urine, sputum, sweat, tears, cervical secretion, and amniotic fluid. In the case of body fluids, cell free DNA (primarily from placenta, a fetal tissue) can be used for estimation of risk. In other embodiments, the sample is a tissue sample of a patient. Examples of tissue samples include placental tissue or fetal cells from amniotic fluid.

In embodiments, the methylation sites are used in many different combinations to calculate the probability of CP in an individual.

In embodiments, the patient is an embryo or fetus. The patient is a newborn or a pediatric patient. In embodiments, when the patient is an embryo or fetus, maternal body fluid can also be used to obtain DNA, especially cfDNA, in the method described herein to predict and/or diagnose the patient for CP or to predict the risk of the patient for having CP.

In embodiments, the disclosure describes determining the risk or predisposition to having a CP at any time during any period of postnatal life. This would involve taking blood, buccal swab or other sources of DNA samples from a newborn or a child.

In embodiments, the DNA is obtained from cells. In embodiments, the DNA is cell free DNA. In embodiments, the DNA is DNA of a fetus obtained from maternal body fluids or placental tissue. The DNA obtained from maternal body fluids can be cell free DNA. In embodiments, the DNA is obtained from amniotic fluid, fetal blood or cord blood obtained at birth.

In embodiments, the sample is obtained and stored for purposes of pathological examination. In embodiments, the sample is stored as slides, tissue blocks, or frozen. In other embodiments, the CP can be any of its subtypes such as Spastic CP, Dyskinetic CP or Ataxic CP.

The present disclosure provides intragenic cytosine markers and their performance as represented by the Area under the ROC curve (AUROC) and 95% Confidence Interval (CI) for the detection of CP versus unaffected controls in Table 1. The CI range that does not cross (i.e. go below) 0.50 indicates statistical significance. Table 2 indicates extra-genic cytosine markers (outside of recognized genes) for CP prediction.

In embodiments, measurement of the frequency or percentage methylation of cytosine nucleotides is obtained using gene or whole genome sequencing techniques.

In another embodiment, the assay is a bisulfite-based methylation assay or DNA methylation sequencing to identify methylation changes in individual cytosines throughout the genome.

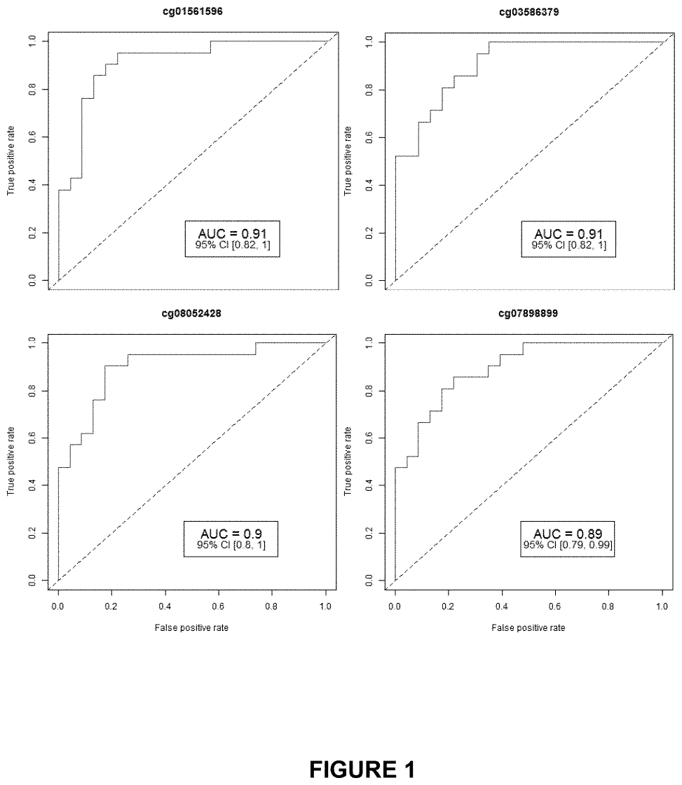

In embodiments, the disclosure describes a method by which proteins transcribed from the genes listed in Table 1 can be measured in body fluids (maternal and affected individuals) and used to detect and distinguish different types of CP. FIG. 1 shows the actual ROC curves for four of these CpG loci (and associated genes).

In embodiments, proteins transcribed from related genes showing DNA methylation changes can be measured and quantitated in body fluids and or tissues of pregnant mothers or affected individuals.

In embodiments, mRNA produced by affected genes showing DNA methylation changes is measured in tissue or body fluids and mRNA levels can be quantitated to determine activity of said genes and used to estimate likelihood of CP. In embodiments, the method further comprises the use of an mRNA genome-wide chip for the measurement of gene activity of genes genome-wide for screening any tissue (including placenta) or body fluids (including blood, amniotic fluid, cervical secretion, and saliva) containing mRNA.

Tables of Genes and Genomic Loci. Table 1, Table 2, and Supplementary Tables S1A-S1E, disclosed in the Examples, provide genomic loci that can be used to predict or diagnose CP in subjects. One or more of the genomic loci in Table 1, Table 2, and Tables S1A-S1E can be selected for predicting, detecting, and/or diagnosing CP in subjects.

Table 1 provides 220 genomic loci. One or more, two or more, three or more, up to and including all 220 of the genomic loci in Table 1 can be selected for predicting, detecting, and/or diagnosing CP in a subject. In embodiments, one or more, two or more, three or more up to and including the first 115 or first 20 genomic loci disclosed in Table 1 can be selected for predicting, detecting, and/or diagnosing CP. In embodiments, exemplary genomic loci providing predictive accuracy for predicting, detecting, and/or diagnosing CP include cg01561596, cg03586379, cg08052428 and cg07898899.

Likewise, one, one or more, two or more, up to and including all of the genomic loci in Table 2 and Supplemental Tables S1A-S1E can be used for predicting, detecting, and/or diagnosing CP in a subject.

In embodiments, the one or more selected genomic loci have an AUC of 0.60, 0.65, 0.70, 0.75, 0.80, 0.85, 0.90, 0.95, 0.96, 0.97, 0.98, or 0.99. Ranges described throughout the application include the specified range, the sub-ranges within the specified range, the individual numbers within the range, and the endpoints of the range. For example, description of a range such as from one or more up to 220 includes subranges such as from one or more to 100 or more, from 10 or more to 20 or more, from one or more to five or more, as well as individual numbers within that range, for example, 1, 2, 3, 4, 5, 10, 20, 100, and 173. Moreover, as further example, the description of a range of ≥0.75 would include all the individual numbers from 0.75 to 1.00 and including 0.75 and 1.0. Computer programs such as “R” program (version 3.2.2.) can be sued to generate AUC for individual CpG loci or combinations of loci.

In embodiments, differentially methylated genes in the blood DNA of newborns of CP include UFM1, SLC25A36, RALGDS, S100A13. In embodiments, the genes associated with CP include ADAM12, FGF8, PTEN, PDE3B, SMAD1, and RUNX3. Moreover, microRNA, miR-1469, is linked with CP.

In embodiments, the eight CpGs for use as markers for predicting, detecting, and/or diagnosing CP include cg12425861, cg19499452, cg08894153, cg24455365, cg13187827, cg12204727, cg03586379, and cg08634464. These eight markers can be used as a combination of one or more, two or more, three or more, four or more, five or more, six or more, seven or more, or all eight for predicting, detecting, and/or diagnosing CP in subjects. The logistic regression analysis for the combination of 8 CpG sites: AUC=1, Sens=100%, Spec=100%, and Accuracy=100% by using eight CpG (selected by mSVM-RFE).

The microarray systems described herein includes one or more genomic loci described in Table 1, 2, and Supplementary Tables S1A-S1E. In embodiments, the microarray systems include at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, or 210 loci of Table 1, 2, and Supplementary Tables S1A-S1E. In embodiments, the microarray systems include one or more of the following loci: cg12425861, cg19499452, cg08894153, cg24455365, cg13187827, cg12204727, cg03586379, or cg08634464. In embodiments, the microarray systems include the following loci: cg12425861, cg19499452, cg08894153, cg24455365, cg13187827, cg12204727, cg03586379, and cg08634464.

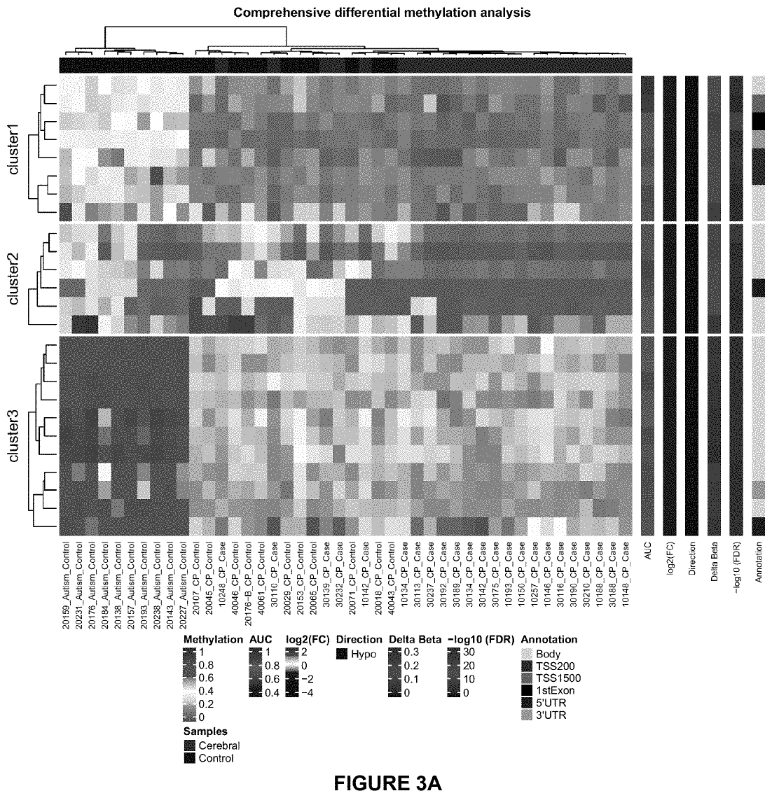

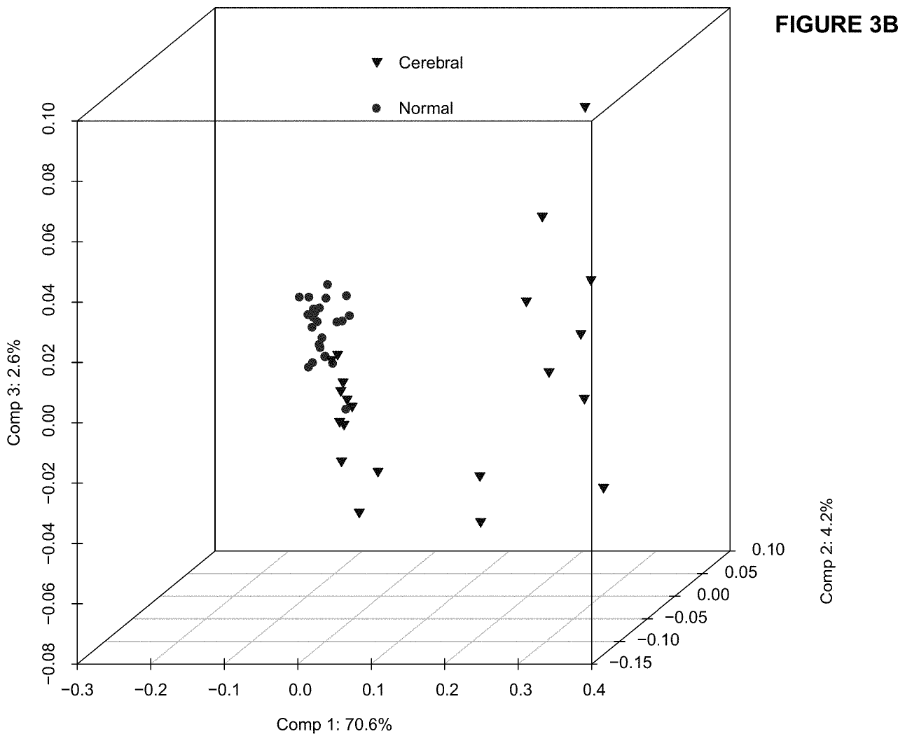

Heat Map. Using the top 25 CpG sites, good discrimination of CP cases from controls was achieved as shown in the Heat Map (FIG. 3A).

Principal Component Analysis. Using three principal components, i.e., features and/or predictive markers in the principal component analysis (PCA), good segregation or clustering of CP cases from controls were achieved (FIG. 3B).

MicroRNA. MicroRNA (miRNA) is an important epigenetic mechanism and exerts control over DNA methylation and suppresses gene expression among other functions. Therefore, the methylation status of known microRNA genes can be measured instead of measuring actual miRNA levels to predict or diagnose CP. Given that DNA methylation status is known to correlate with gene expression, this approach can be used to identify miRNAs that are involved in CP development. miR-1469 was found to be differentially methylated in CP cases. The p value was highly significant, 1.27E-08 (Table S1A). Differential expression of miR-1469 has been observed in neurologic complications such as glioblastoma multiforme, amyotrophic lateral sclerosis, temporal lobe epilepsy, and DiGeorge Syndrome.⁴⁹⁻⁵²

Open Reading Frame. Open Reading Frame (ORF) is typically used for predication of genes whose chromosome mutations are known but have not yet been named. Table S1B shows the values for predicting, detecting, and/or diagnosing CP using ORF. Short non-coding RNA (SNOR) genes for predicting, detecting, and/or diagnosing CP are shown in Table S1C. Non-Coding RNA (NcRNA) genes are shown in Table S1D) for predicting, detecting, and/or diagnosing CP, and genes of uncertain functions (LOC) are shown in Table S1E for predicting, detecting, and/or diagnosing CP.

Kits. Kits for predicting, detecting, and/or diagnosing CP are described. The kits can include all the components for extracting nucleic acid including DNA from the subject, of the microarray system, and/or for analysis of the differentially methylated genomic sites. The microarray system includes the one or more biomarkers described above, for examples, those in Table 1, 2, and Supplementary Tables S1A-S1E. In embodiments, the microarray systems include one or more of the following loci: cg12425861, cg19499452, cg08894153, cg24455365, cg13187827, cg12204727, cg03586379, or cg08634464. In embodiments, the microarray systems include the following loci: cg12425861, cg19499452, cg08894153, cg24455365, cg13187827, cg12204727, cg03586379, and cg08634464.

Treatments. Treatments depends on the type of CP the subject. Treatment can include therapies such as physical therapy including the use of orthotics, medication, surgery, and alternative medicine.

Therapies include physical therapy, occupational therapy, speech and language therapy, and recreational therapy.

Medication can help manage certain conditions such as seizure, involuntary movement, spasticity, incontinence, and gastroesophageal reflux. Medications include muscle or nerve injections and oral muscle relaxants. Muscle or nerve injections such as onabotulinumtoxin A (Botox, Dysport) can be used to treat tightening of a specific muscle. Oral muscle relaxants including diazepam (Valium), dantrolene (Dantrium), baclofen (Gablofen, Lioresal) and tizanidine (Zanaflex) can be used to relax muscles.

Surgery can help correct movement problems and improve mobility in children with CP, for example spastic CP. Orthopedic surgery can correct severe contractures or deformities on bones or joints to place arms, hips, or legs in their correct positions. Orthopedic surgery can also lengthen muscles and tendons that are shorted by contractures. Selective dorsal rhizotomy (cutting nerve fibers) can be performed in severe cases to cut the nerves serving the spastic muscles.

Alternative medicine, though not accepted in clinical practice, have been used to treat CP. An example of alternative medicine includes hyperbaric oxygen therapy.

Uniqueness of Epigenetic Approach. What is unique about the disclosure, among other features, is the fact that the epigenetic changes can be identified and monitored in perpheral leucocyte (blood DNA) and not only in brain tissue. This is important as the latter is only available, for all intents and purposes, except in post-mortem specimens. The use of blood leucocyte DNA is based on the finding that the same environmental factors that induce epigenetic changes in the brain and thereby lead to cerebral palsy (CP) induce some similar, related or parallel epigenetic changes in the genes of leucocyte DNA. This hypothesis is consistent with mounting evidence that DNA methylation status of peripheral cells, most particularly from leucocyte, may be useful for the detection of brain disorders.

Methods disclosed herein include treating subjects and individuals who are patients that are in need of prediction of risk, diagnosis, and/or treatment of CP. Patients includes mammals such as human. Patients also include embryo and fetus. Subjects in need of a treatment or diagnosis (or subject in need thereof) are patients having symptoms of CP or patients that are in need of being screened or tested for CP.

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

In addition, unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±15% of the stated value; ±10% of the stated value; ±5% of the stated value; ±4% of the stated value; ±3% of the stated value; ±2% of the stated value; ±1% of the stated value; or ±any percentage between 1% and 20% of the stated value.

The terms “a,” “an,” “the” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The following examples illustrate exemplary methods provided herein. These examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXEMPLARY EMBODIMENTS

The following are Exemplary Embodiments:

1. A method for predicting, detecting, and/or diagnosing cerebral palsy (CP), wherein the method includes:
obtaining a sample from the patient;
extracting nucleic acid from the sample;
assaying the nucleic acid to determine a frequency or percentage methylation of cytosine at one or more loci throughout genome; and
comparing the cytosine methylation level of the patient to a well characterized population of normal or unaffected controls and cerebral palsy groups.

2. The method of embodiment 1, wherein the method further includes calculating the individual risk of CP based on the cytosine methylation level at different sites throughout the genome.

3. The method of embodiment 1 or 2, wherein the nucleic acid is cell free DNA obtained from body fluid or cellular DNA obtained from a tissue of the patient.

4. The method of any one of embodiments 1-3, wherein the sample is blood, plasma, serum, urine, saliva, sputum, amniotic fluid, cervical fluid or secretion, urine, tear, sweat, placental tissue, or a buccal swab.

5. The method of any one of embodiments 1-4, wherein the percentage methylation of cytosines are determined for different combinations of loci to calculate the probability of CP in an individual.

6. The method of any one of embodiments 1-5, wherein the patient is a fetus or embryo, newborn, or pediatric patient.

7. The method of any one of embodiments 1-6, wherein the DNA is obtained from cells.

8. The method of any one of embodiments 1-6, wherein the DNA is cell free and extracted from body fluid.

9. The method of any one of embodiments 1-8, wherein the DNA is DNA of a fetus or embryo obtained from maternal body fluids or placental tissue.

10. The method of any one of embodiments 1-9, wherein the DNA is obtained from amniotic fluid, fetal blood, or cord blood obtained at birth.

11. The method of any one of embodiments 1-10, wherein the one or more loci include at least two, three, four, five, six, seven, eight, nine, ten, fifteen, twenty, twenty-five, thirty, forty, or fifty loci.

12. The method of any one of embodiments 1-11, wherein the one or more loci is selected from Table 1.

13. The method of any one of embodiments 1-12, wherein the one or more loci is selected from Table 1 and has an AUC of 0.75 or greater, 0.80 or greater, 0.85 or greater, 0.90 or greater, or 0.95 or greater.

14. The method of any one of embodiments 1-13, wherein the one or more loci are selected from Table S1A, Table S1 B, Table S1C, Table S1 D, or Table S1E.

15. The method of any one of embodiments 1-14, wherein the assay is a bisulfite-based methylation assay or a whole genome methylation assay.

16. The method of any one of embodiments 1-15, wherein measurement of the frequency or percentage methylation of cytosine nucleotides is obtained using gene or whole genome sequencing techniques.

17. The method of any one of embodiments 1-16, wherein the sample is obtained and stored for purposes of pathological examination.

18. The method of embodiment 17, wherein the sample is stored as slides, tissue blocks, or frozen.

19. The method of any one of embodiments 1-18, wherein the method further comprises extracting RNA from the sample; assaying the expression of one or more transcripts of the RNA sample, wherein the one or more transcripts are transcripts that are regulated by methylation of a CpG locus that is differentially methylated in CP cases as compared to non-CP cases; and comparing expression level of the one or more transcripts of the RNA sample to a well characterized population of normal group and/or cerebral palsy group.

20. The method of any one of embodiments 1-19, wherein the method further comprises extracting one or more proteins from the sample; assaying expression of one or more proteins in the protein sample, wherein the proteins are proteins with expression regulated by methylation of a CpG locus that is differentially methylated in CP cases as compared to non-CP cases; and
comparing expression level of one or more proteins in the protein sample to a well characterized population of normal group and/or cerebral palsy group. 21. A method of predicting, detecting, and/or diagnosing CP in a patient including:
obtaining a sample from the patient;
extracting RNA from the sample of the patient;
assaying the expression of one or more transcripts of the RNA sample, wherein the one or more transcripts are transcripts that are regulated by methylation of a CpG locus that is differentially methylated in CP cases as compared to non-CP cases; and
comparing expression level of the one or more transcripts of the RNA sample to a well characterized population of normal group and/or cerebral palsy group.

22. The method of embodiment 21, wherein the method further includes calculating the patient's risk of CP based on the expression level of the one or more transcripts.

23. The method of embodiment 21 or 22, wherein the RNA is miRNA or mRNA.

24. The method of any one of embodiments 21-23, wherein the sample includes tissue or body fluid of the patient.

25. A method for predicting, detecting, and/or diagnosing CP, wherein mRNA produced by affected genes (genes that have a change in methylation) is measured in tissue or body fluids and mRNA levels can be quantitated to determine activity of said genes and used to estimate likelihood of CP.

26. The method of any one of embodiments 1-25, further including the use of an mRNA genome-wide chip for the measurement of gene activity of genes genome-wide for screening the biological sample.

27. A method of predicting, detecting, and/or diagnosing CP in a patient including:
obtaining a sample from a patient;
extracting one or more proteins from the sample;
assaying expression of one or more proteins in the protein sample, wherein the proteins include proteins with expression regulated by methylation of a CpG locus that is differentially methylated in CP cases as compared to non-CP cases; and
comparing expression level of one or more proteins in the protein sample to a well characterized population of normal group and/or cerebral palsy group.

28. The method of embodiment 27, wherein the method further includes calculating the patient's risk of CP based on the expression level of the one or more proteins.

29. The method of embodiment 27 or 28, wherein the sample includes tissue or body fluid of the patient.

30. The method of any one of embodiments 27-29, further including determining the risk or predisposition to having a CP at any time during any period of postnatal life.

31. The method of any one of embodiments 1-30, wherein the method further includes treating the patient postnatally.

32. The method of any one of embodiments 1-31, wherein the method further includes treating the patient postnatally by therapy, medication, and/or surgery to correct the defect.

33. The method of any one of embodiments 1-32, wherein the method includes using microarray chips designed to determine CpG methylation of genes known and suspected to be involved in brain neurological and neuromotor development and function that will optimize the prediction of CP and the different types of CP.

34. The method of any one of embodiments 1-33, wherein the one or more loci include one or more of cg12425861, cg19499452, cg08894153, cg24455365, cg13187827, cg12204727, cg03586379, or cg08634464.

35. The method of any one of embodiments 1-34, wherein the one or more loci include cg12425861, cg19499452, cg08894153, cg24455365, cg13187827, cg12204727, cg03586379, and cg08634464.

36. The method of any one of embodiments 1-35, wherein the method further includes performing logistic regression.

37. The method of any one of embodiments 1-36, wherein the method further includes performing deep learning and/or machine learning algorithms.

38. A microarray including one or more nucleic acids, wherein the one or more nucleic acids include one or more genomic loci selected from Table 1.

39. The microarray of embodiment 38, wherein the nucleic acids include at least two, three, four, five, six, seven, eight, nine, ten, fifteen, twenty, twenty-five, thirty, forty, fifty, sixty, seventy, eighty, ninety, or one hundred loci.

40. The microarray of embodiments 38 or 39, wherein the one or more loci include one or more of cg12425861, cg19499452, cg08894153, cg24455365, cg13187827, cg12204727, cg03586379, or cg08634464.

41. The microarray of any one of embodiments 38-40, wherein the loci include cg12425861, cg19499452, cg08894153, cg24455365, cg13187827, cg12204727, cg03586379, and cg08634464.

42. A microarray including one or more nucleic acids, wherein the one or more nucleic acids include one or more genomic loci of cg12425861, cg19499452, cg08894153, cg24455365, cg13187827, cg12204727, cg03586379, or cg08634464.

43. The microarray of embodiment 42, wherein the one or more nucleic acids include at least two, three, four, five, six, seven, or eight of the loci.

44. The microarray of embodiment 42 or 43, wherein the loci include cg12425861, cg19499452, cg08894153, cg24455365, cg13187827, cg12204727, cg03586379, and cg08634464.

### EXAMPLES

### Example 1

It was hypothesized that genome-wide epigenetic alterations can be detected in newborn blood DNA in association with CP. A genome-wide DNA methylation analysis was conducted using Illumina HumanMethylation450K arrays in 23 CP cases relative to 21 normal controls. Comparison of the methylation profiles between CP and control subjects revealed 220 differentially methylated individual CpG loci associated with 220 independent genes that had a greater than 10% difference in methylation (false discovery rate (FDR) P≤0.05) with a mean β-value difference of ≥0.2 (at least 2.0-fold). These CpG sites were limited to cases with reasonable good to excellent predictive accuracy, i.e. they have a receiver operating curve area under the curve (ROC AUC) ≥0.75 for CP detection. The array data was validated by bisulphite pyrosequencing. Gene ontology and pathway analysis was performed by Qiagen's Ingenuity Pathway Analysis (IPA). This determines whether the genes identified have biological plausibilities. IPA identified multiple canonical pathways associated with CP. The ten pathways enriched among the differentially methylated CpGs included Axonal guidance and Actin cytoskeleton signaling, Wnt-signaling, Insulin receptor and PI3K/AKT signaling, TGF-B signaling, Crosstalk between Dendritic Cells and Natural Killer Cells, Neuroinflammation Signaling Pathway, Ephrin Receptor Signaling, Neuregulin Signaling and Tight Junction Signaling. Multiple genes known for their involvement in biological processes and functions related to CP development, including: neuromotor damage, malformation of major brain structures, brain growth, neuroprotection, neuronal development and dedifferentiation, and cranial sensory neuron development. Some of the identified genes are ADAM12, FGF8, PTEN, PDE3B, SMAD1, RUNX3 as well as miR-1469. Thus, many of the genes identified are known to play a role in brain and neuromotrr function which are adversely affected in CP suggesting that the findings have biological plausibility. For the first time, significant discrete methylation changes prior to the onset of clinical CP manifestation were identified. They can be useful as biomarkers for early therapeutic intervention.

In the current study, global methylation profiling of CP cases and normal controls were analyzed using HumanMethylation450K bead chips. After analysis of the methylation differences and then in combination with gene network analysis using Ingenuity® Pathway Analysis (IPA), a set of genes that were deregulated by aberrant DNA methylation in CP was identified. 220 aberrant DNA methylation genes were selected for further analysis based on AUC ROC (AUC≥0.75), 2-fold change, p-values (0.05) and % of methylation (≥10%), with validation analysis using additional CP subjects and normal controls.

Materials and methods. Differential Methylation Assay: CpGs showing differential methylation in CP relative to normal controls were identified using the Illumina HumanMethylation450K arrays. Genomic DNA from archived blood spots was isolated using Puregene DNA Purification kits (Gentra systems® MN, USA) according to manufacturer's protocols. Newborn blood spot specimens were provided by the Michigan Department of Community Health in the State of Michigan (MDCH) and leftover samples used. The samples were collected previously for the mandated newborn screening and treatment program run by MDCH. All specimens were collected between 24 and 79 hours after birth. Parents/legal guardians of child provided informed consent. The Institutional Review Boards from both Wayne State University and the Michigan Department of Community Health approved this study. The DNA samples were bisulfite converted using the EZ DNA Methylation-Direct Kit (Zymo Research, Orange, Calif.) per the manufacturer's protocol and processed according to Illumina protocols for HumanMethylation450K arrays.

Epigenome-wide methylation scan using the Illumina. HumanMethylation450K arrays. Genome wide methylation analysis was conducted on CP and control samples using the human 450,000 methylation sites. The processing was done as per manufacturer's protocol. Fluorescently stained BeadChips were imaged by the Illumina iScan, following a series of stringent quality control and filtering criteria, as described previously.⁴⁹

Statistical and Bioinformatic analysis. Bioinformatic and statistical analysis, data preprocessing and quality control was performed, including examination of the background signal intensity of both CP subjects and normal controls. DNA methylation was measured using the Genome Studio methylation analysis package (Illumina). DNA methylation β-value (level of cytosine or CpG locus methylation) was assigned to each CpG site. Differential methylation was assessed by comparing the β-values per individual nucleotide at each CpG site between cases and controls. Confounding factors such as probes associated with sex chromosomes and SNPs in the probe sequence (listing dbSNP entries within 10 bp of the CpG site) were removed for further analysis as the probe sequence may influence corresponding methylated probes.

Based on pre-set cutoff criteria for probes with ≥2.0-fold increase and/or ≥2.0-fold decrease with False Discovery Rate (FDR) p<0.05, AUC ROC≥0.75 and 10% methylations variation were considered for further network and pathway analysis.

The identified differentially-methylated genes were used to generate a heatmap using the ComplexHeatmap (v1.6.0) R package (v3.2.2). Ward distance was used for the hierarchical clustering of samples. Only genes for which Entrez identifiers were further analyzed. QIAGEN′S Ingenuity Pathway Analysis (IPA) (Qiagen IPA) software was used to identify biological functions or interacting canonical pathways. Over-represented canonical pathways, biological processes and molecular processes was identified.

Identification of differential methylation between CP and normal controls. To explore the CP whole-genome DNA methylation, 23 blood DNA samples from CP subjects and 2 from controls were analyzed using the Illumina HumanMethylation450K array. The detailed clinical data was presented in Table 1. After quality control and filtering, by using various statistical approaches. A total of 220 genes were found to be differentially methylated with FDR p<0.05, irrespective of AUC. However, 220 CpGs were found to have a statistically significantly different DNA methylation status between CP and controls (False Detection Rate (FDR) p-value<0.05) compared to controls and in addition had high predictive accuracy for diagnosing CP (area under the receiver operating characteristics curve (ROC AUC)≥0.75). A total of 219 CpGs were hypomethylated in CP (Table 1), and one with hypermethylation was detected. Among these, the maximum number of altered CpGs were in the gene body followed by 5′UTR, 1^{st} exon, TSS200, TSS1500 and 3′UTR.

TABLE 1
Details of each target significantly differentially methylated in CP. Target ID, Gene ID, chromosome location, %
methylation change and FDR p-value.

| | | | | | % | | | | | |
|---|---|---|---|---|---|---|---|---|---|---|
| | | | | % Methylation | Methylation | | | | | |
| Index | TargetID | CHR | Gene | Cases | Control | Fold change | FDR p-Val | AUC | CI_lower | CI_upper |
| 32308 | cg01561596 | 13 | UFM1 | 1.568 | 3.673 | 0.427 | 0.002962249 | 0.911 | 0.819 | 1.000 |
| 72540 | cg03586379 | 3 | SLC25A36 | 2.332 | 5.643 | 0.413 | 1.01991E−05 | 0.909 | 0.816 | 1.000 |
| 156309 | cg08052428 | 9 | RALGDS | 4.659 | 9.627 | 0.484 | 1.53312E−08 | 0.901 | 0.804 | 0.998 |
| 153567 | cg07898899 | 1 | S100A13 | 7.107 | 16.869 | 0.421 | 3.71708E−20 | 0.894 | 0.794 | 0.994 |
| 365798 | cg20376421 | 12 | MYL6B | 4.142 | 8.413 | 0.492 | 4.40443E−07 | 0.884 | 0.780 | 0.989 |
| 314131 | cg17142950 | 1 | SAMD13 | 12.209 | 27.607 | 0.442 | 1.32642E−30 | 0.878 | 0.771 | 0.985 |
| 194868 | cg10230427 | 6 | BAG2 | 4.224 | 10.243 | 0.412 | 6.69602E−12 | 0.870 | 0.759 | 0.980 |
| 266675 | cg14347670 | 6 | CCND3 | 2.808 | 7.067 | 0.397 | 5.68407E−08 | 0.865 | 0.753 | 0.978 |
| 369741 | cg20640432 | 19 | CREB3L3 | 2.910 | 5.855 | 0.497 | 0.000148195 | 0.865 | 0.753 | 0.978 |
| 228110 | cg12204727 | 15 | COMMD4 | 1.630 | 3.273 | 0.498 | 0.02176129 | 0.860 | 0.746 | 0.974 |
| 223966 | cg11961138 | 17 | IGFBP4 | 6.143 | 15.870 | 0.387 | 2.48421E−21 | 0.857 | 0.742 | 0.972 |
| 228141 | cg12206423 | 13 | SLITRK5 | 2.914 | 5.903 | 0.494 | 0.000118856 | 0.857 | 0.742 | 0.972 |
| 373355 | cg20871904 | 4 | YTHDC1 | 2.752 | 5.916 | 0.465 | 3.951E−05 | 0.857 | 0.742 | 0.972 |
| 10016 | cg00472801 | 6 | KHDRBS2 | 4.085 | 8.230 | 0.496 | 8.39989E−07 | 0.855 | 0.739 | 0.971 |
| 66943 | cg03307401 | 19 | KLK13 | 1.451 | 4.086 | 0.355 | 0.000174134 | 0.855 | 0.739 | 0.971 |
| 325395 | cg17852224 | 22 | MAPK8IP2 | 5.512 | 11.832 | 0.466 | 1.45237E−11 | 0.855 | 0.739 | 0.971 |
| 466038 | cg26707202 | 4 | SMAD1 | 2.662 | 6.349 | 0.419 | 1.68449E−06 | 0.855 | 0.739 | 0.971 |
| 56688 | cg02782426 | 3 | ENTPD3 | 3.905 | 8.256 | 0.473 | 1.93735E−07 | 0.853 | 0.736 | 0.970 |
| 283125 | cg15277906 | 8 | GDF6 | 2.503 | 5.053 | 0.495 | 0.000734586 | 0.851 | 0.733 | 0.969 |
| 399434 | cg22624212 | 21 | WDR4 | 1.747 | 4.042 | 0.432 | 0.001372057 | 0.851 | 0.733 | 0.969 |
| 423143 | cg24069733 | 20 | DBNDD2; SYS1- | 1.749 | 4.094 | 0.427 | 0.001070153 | 0.847 | 0.728 | 0.966 |
| | | | DBNDD2 | | | | | | | |
| 372561 | cg20810398 | 1 | EXOSC10 | 1.265 | 2.641 | 0.479 | 0.049498898 | 0.847 | 0.728 | 0.966 |
| 69411 | cg03433549 | 12 | PA2G4 | 1.855 | 3.908 | 0.475 | 0.004561501 | 0.847 | 0.728 | 0.966 |
| 172273 | cg08931196 | 11 | RNF26 | 1.326 | 2.811 | 0.472 | 0.034503544 | 0.847 | 0.728 | 0.966 |
| 22518 | cg01067849 | 6 | WRNIP1 | 1.761 | 4.229 | 0.417 | 0.00058363 | 0.847 | 0.728 | 0.966 |
| 405620 | cg23000734 | 10 | CTBP2 | 8.083 | 17.708 | 0.456 | 1.39532E−18 | 0.845 | 0.725 | 0.965 |
| 196650 | cg10333402 | 7 | MOGAT3 | 5.085 | 10.347 | 0.491 | 5.14432E−09 | 0.845 | 0.725 | 0.965 |
| 358844 | cg19917744 | 2 | PLEKHM3 | 2.319 | 6.023 | 0.385 | 8.95009E−07 | 0.845 | 0.725 | 0.965 |
| 106002 | cg05332869 | 20 | TOP1 | 2.784 | 5.691 | 0.489 | 0.000159202 | 0.845 | 0.725 | 0.965 |
| 35112 | cg01712673 | 17 | WBP2 | 1.928 | 3.915 | 0.492 | 0.006349591 | 0.843 | 0.722 | 0.963 |
| 158632 | cg08171351 | 22 | CECR6 | 4.571 | 9.405 | 0.486 | 2.98587E−08 | 0.841 | 0.719 | 0.962 |
| 66994 | cg03309770 | 16 | FAM18A | 5.597 | 11.549 | 0.485 | 1.80402E−10 | 0.841 | 0.719 | 0.962 |
| 319890 | cg17486946 | 10 | FGF8 | 3.330 | 7.320 | 0.455 | 7.20495E−07 | 0.841 | 0.719 | 0.962 |
| 334214 | cg18384060 | 10 | PTEN; KILLIN | 1.459 | 3.150 | 0.463 | 0.016687893 | 0.841 | 0.719 | 0.962 |
| 336511 | cg18516195 | 14 | BEGAIN | 11.677 | 25.730 | 0.454 | 8.53915E−28 | 0.839 | 0.717 | 0.960 |
| 322627 | cg17674287 | 6 | BRD2 | 1.277 | 2.741 | 0.466 | 0.036359097 | 0.839 | 0.717 | 0.960 |
| 330104 | cg18132212 | 4 | NSUN7 | 1.256 | 2.919 | 0.430 | 0.016798353 | 0.839 | 0.717 | 0.960 |
| 296816 | cg16126458 | 1 | AKR7A3 | 2.656 | 5.916 | 0.449 | 2.05915E−05 | 0.836 | 0.714 | 0.959 |
| 370364 | cg20677058 | 1 | AKR7L | 4.155 | 9.968 | 0.417 | 2.37806E−11 | 0.834 | 0.711 | 0.958 |
| 334950 | cg18426487 | 10 | CUL2 | 1.651 | 3.658 | 0.451 | 0.004898452 | 0.834 | 0.711 | 0.958 |
| 106572 | cg05359249 | 2 | CHPF | 1.048 | 2.695 | 0.389 | 0.016150517 | 0.832 | 0.708 | 0.956 |
| 188686 | cg09883524 | 16 | MC1R | 1.534 | 3.269 | 0.469 | 0.014501199 | 0.832 | 0.708 | 0.956 |
| 161115 | cg08301299 | 16 | RNPS1 | 3.292 | 8.126 | 0.405 | 3.08386E−09 | 0.832 | 0.708 | 0.956 |
| 347592 | cg19243130 | 11 | SIAE; SPA17 | 2.080 | 4.557 | 0.456 | 0.000736722 | 0.832 | 0.708 | 0.956 |
| 311960 | cg17009717 | 2 | POLR1B | 1.637 | 3.318 | 0.493 | 0.018851112 | 0.830 | 0.705 | 0.955 |
| 51992 | cg02553987 | 17 | BCAS3 | 1.317 | 2.884 | 0.457 | 0.025263275 | 0.828 | 0.703 | 0.954 |
| 246992 | cg13404674 | 12 | IQSEC3 | 24.547 | 49.449 | 0.496 | 2.48906E−28 | 0.828 | 0.703 | 0.954 |
| 120193 | cg06106763 | 21 | OLIG1 | 1.062 | 3.527 | 0.301 | 0.000296879 | 0.828 | 0.703 | 0.954 |
| 24413 | cg01158970 | 5 | UTP15; | 1.819 | 3.930 | 0.463 | 0.003434011 | 0.828 | 0.703 | 0.954 |
| | | | ANKRA2 | | | | | | | |
| 475379 | cg27253814 | 7 | ZNF789 | 1.894 | 3.901 | 0.485 | 0.005689183 | 0.828 | 0.703 | 0.954 |
| 2643 | cg00114084 | 1 | AK2 | 1.163 | 2.827 | 0.411 | 0.01594852 | 0.826 | 0.700 | 0.952 |
| 245621 | cg13331200 | 3 | CADM2 | 2.745 | 6.650 | 0.413 | 4.95689E−07 | 0.826 | 0.700 | 0.952 |
| 293925 | cg15953602 | 8 | CRISPLD1 | 2.072 | 4.238 | 0.489 | 0.003174684 | 0.826 | 0.700 | 0.952 |
| 3750 | cg00167275 | 10 | FAM35A; | 8.002 | 17.565 | 0.456 | 1.72636E−18 | 0.826 | 0.700 | 0.952 |
| | | | GLUD1 | | | | | | | |
| 90716 | cg04527840 | 4 | GAR1 | 1.219 | 2.919 | 0.418 | 0.014187856 | 0.826 | 0.700 | 0.952 |
| 203834 | cg10760299 | 15 | GATM | 8.323 | 16.752 | 0.497 | 6.43649E−15 | 0.826 | 0.700 | 0.952 |
| 55892 | cg02743650 | 11 | IGSF22 | 3.804 | 7.611 | 0.500 | 3.9664E−06 | 0.826 | 0.700 | 0.952 |
| 197519 | cg10384919 | 22 | MEI1 | 4.501 | 9.485 | 0.474 | 1.06101E−08 | 0.826 | 0.700 | 0.952 |
| 140071 | cg07162198 | 20 | SLC2A10 | 1.883 | 3.834 | 0.491 | 0.007186509 | 0.826 | 0.700 | 0.952 |
| 173098 | cg08979136 | 5 | TRIM36 | 1.143 | 2.567 | 0.445 | 0.039867394 | 0.826 | 0.700 | 0.952 |
| 468363 | cg26842664 | 18 | ZNF397 | 2.123 | 4.789 | 0.443 | 0.000292399 | 0.826 | 0.700 | 0.952 |
| 32561 | cg01572696 | 4 | IDUA | 6.444 | 13.080 | 0.493 | 1.21401E−11 | 0.824 | 0.697 | 0.951 |
| 210438 | cg11156873 | 5 | LPCAT1 | 13.168 | 29.158 | 0.452 | 1.88475E−30 | 0.824 | 0.697 | 0.951 |
| 107240 | cg05389183 | 5 | PPIC | 4.620 | 9.670 | 0.478 | 8.68106E−09 | 0.824 | 0.697 | 0.951 |
| 78 | cg00003287 | 1 | TNNT2 | 2.716 | 5.904 | 0.460 | 3.30877E−05 | 0.824 | 0.697 | 0.951 |
| 450545 | cg25781121 | 3 | ZNF589 | 1.451 | 2.993 | 0.485 | 0.02941221 | 0.824 | 0.697 | 0.951 |
| 257949 | cg13931999 | 9 | HINT2 | 1.663 | 3.735 | 0.445 | 0.003681915 | 0.822 | 0.695 | 0.949 |
| 126179 | cg06463589 | 16 | MT1E | 1.614 | 3.340 | 0.483 | 0.015689583 | 0.822 | 0.695 | 0.949 |
| 272260 | cg14621053 | 10 | ADAM12 | 1.509 | 3.155 | 0.478 | 0.020354424 | 0.820 | 0.692 | 0.948 |
| 253649 | cg13717541 | 14 | CLMN | 23.048 | 49.485 | 0.466 | 5.38429E−28 | 0.818 | 0.689 | 0.947 |
| 236242 | cg12721730 | 13 | PCDH20 | 3.586 | 7.795 | 0.460 | 2.79951E−07 | 0.818 | 0.689 | 0.947 |
| 135795 | cg06951245 | 2 | PTH2R | 2.778 | 6.189 | 0.449 | 1.01565E−05 | 0.818 | 0.689 | 0.947 |
| 243580 | cg13206850 | 7 | ATXN7L1 | 20.642 | 41.312 | 0.500 | 2.6793E−29 | 0.816 | 0.686 | 0.945 |
| 54586 | cg02678768 | 17 | EVPL | 19.753 | 42.111 | 0.469 | 6.63899E−29 | 0.816 | 0.686 | 0.945 |
| 308583 | cg16783819 | 6 | HSF2 | 2.126 | 4.506 | 0.472 | 0.001225282 | 0.814 | 0.684 | 0.944 |
| 171103 | cg08867893 | 10 | ZNF365 | 1.570 | 3.416 | 0.459 | 0.009330786 | 0.814 | 0.684 | 0.944 |
| 383881 | cg21558545 | 12 | LGR5 | 2.313 | 5.069 | 0.456 | 0.000220894 | 0.812 | 0.681 | 0.942 |
| 195068 | cg10241347 | 10 | FAM24B | 5.783 | 13.595 | 0.425 | 1.0669E−15 | 0.812 | 0.681 | 0.942 |
| 307908 | cg16741308 | 22 | PARVB | 1.264 | 2.751 | 0.460 | 0.033326936 | 0.812 | 0.681 | 0.942 |
| 264369 | cg14234406 | 8 | PLEC1 | 6.614 | 15.192 | 0.435 | 4.26781E−17 | 0.812 | 0.681 | 0.942 |
| 60503 | cg02970551 | 1 | RUNX3 | 3.408 | 7.783 | 0.438 | 7.59829E−08 | 0.812 | 0.681 | 0.942 |
| 304823 | cg16579438 | 3 | THRB | 3.125 | 7.313 | 0.427 | 1.54209E−07 | 0.812 | 0.681 | 0.942 |
| 364405 | cg20282550 | 10 | AKR1E2 | 3.406 | 9.417 | 0.362 | 9.299E−13 | 0.810 | 0.678 | 0.941 |
| 347328 | cg19226007 | 17 | C1QL1 | 1.730 | 3.911 | 0.442 | 0.002333817 | 0.810 | 0.678 | 0.941 |
| 312000 | cg17012160 | 1 | FMN2 | 3.186 | 6.937 | 0.459 | 2.42695E−06 | 0.810 | 0.678 | 0.941 |
| 309682 | cg16857181 | 7 | KBTBD2 | 2.461 | 5.118 | 0.481 | 0.000418213 | 0.810 | 0.678 | 0.941 |
| 219328 | cg11701583 | 12 | NDUFA4L2 | 9.754 | 23.373 | 0.417 | 7.02363E−29 | 0.810 | 0.678 | 0.941 |
| 207220 | cg10961700 | 1 | SETDB1 | 2.266 | 4.574 | 0.495 | 0.001913219 | 0.810 | 0.678 | 0.941 |
| 410431 | cg23279355 | 5 | CMYA5 | 10.705 | 23.558 | 0.454 | 2.93604E−25 | 0.807 | 0.676 | 0.939 |
| 183932 | cg09605254 | 8 | FAM91A1 | 3.369 | 7.902 | 0.426 | 2.59349E−08 | 0.807 | 0.676 | 0.939 |
| 377464 | cg21144587 | 2 | GPN1; | 6.360 | 12.902 | 0.493 | 1.86136E−11 | 0.807 | 0.676 | 0.939 |
| | | | CCDC121 | | | | | | | |
| 417766 | cg23731836 | 8 | KIF13B | 1.808 | 3.858 | 0.469 | 0.004471214 | 0.807 | 0.676 | 0.939 |
| 392348 | cg22130262 | 8 | MOS | 1.867 | 4.580 | 0.408 | 0.000176656 | 0.807 | 0.676 | 0.939 |
| 36939 | cg01802975 | 1 | SLC35D1 | 2.862 | 5.781 | 0.495 | 0.000162139 | 0.807 | 0.676 | 0.939 |
| 458423 | cg26273962 | 10 | SORBS1 | 0.748 | 2.084 | 0.359 | 0.047063253 | 0.807 | 0.676 | 0.939 |
| 31754 | cg01534217 | 3 | FOXP1 | 1.705 | 4.361 | 0.391 | 0.000202863 | 0.805 | 0.673 | 0.938 |
| 394598 | cg22284043 | 13 | GPC5 | 2.578 | 5.160 | 0.500 | 0.000672636 | 0.805 | 0.673 | 0.938 |
| 402295 | cg22803211 | 4 | OCIAD1 | 1.469 | 3.070 | 0.479 | 0.023823777 | 0.805 | 0.673 | 0.938 |
| 304543 | cg16565409 | 17 | RPL23A | 15.665 | 36.195 | 0.433 | 2.48296E−29 | 0.805 | 0.673 | 0.938 |
| 408262 | cg23161317 | 6 | ZNF389 | 1.193 | 2.796 | 0.427 | 0.020722776 | 0.805 | 0.673 | 0.938 |
| 126986 | cg06508976 | 9 | IER5L | 1.911 | 4.463 | 0.428 | 0.000431147 | 0.803 | 0.670 | 0.936 |
| 196042 | cg10301338 | 18 | KCTD1 | 1.613 | 3.487 | 0.463 | 0.008537725 | 0.803 | 0.670 | 0.936 |
| 220980 | cg11796565 | 19 | NFIX | 3.041 | 6.534 | 0.465 | 8.832E−06 | 0.803 | 0.670 | 0.936 |
| 91795 | cg04582164 | 3 | RAP2B | 2.072 | 4.148 | 0.500 | 0.004742234 | 0.803 | 0.670 | 0.936 |
| 334187 | cg18382422 | 10 | TSPAN15 | 1.864 | 3.973 | 0.469 | 0.003577784 | 0.803 | 0.670 | 0.936 |
| 445648 | cg25465019 | 1 | LMO4 | 0.556 | 2.694 | 0.206 | 0.001083682 | 0.802 | 0.669 | 0.936 |
| 161571 | cg08326511 | 2 | DBI | 1.398 | 2.924 | 0.478 | 0.03057326 | 0.801 | 0.668 | 0.935 |
| 172220 | cg08928494 | 16 | CA5A | 18.858 | 41.326 | 0.456 | 7.04123E−29 | 0.801 | 0.668 | 0.935 |
| 224014 | cg11963883 | 10 | DDX21 | 0.827 | 2.523 | 0.328 | 0.011535854 | 0.801 | 0.668 | 0.935 |
| 100578 | cg05044431 | 5 | GABRA1 | 1.499 | 3.260 | 0.460 | 0.012857159 | 0.801 | 0.668 | 0.935 |
| 151051 | cg07755735 | 2 | GDF7 | 6.813 | 14.079 | 0.484 | 4.64627E−13 | 0.801 | 0.668 | 0.935 |
| 429246 | cg24455365 | 1 | PINK1 | 3.737 | 7.890 | 0.474 | 4.91923E−07 | 0.801 | 0.668 | 0.935 |
| 352953 | cg19580633 | 5 | RPL26L1 | 1.480 | 3.564 | 0.415 | 0.003063357 | 0.801 | 0.668 | 0.935 |
| 155730 | cg08019195 | 11 | SCN4B | 1.439 | 3.106 | 0.463 | 0.018182107 | 0.801 | 0.668 | 0.935 |
| 373900 | cg20914370 | 7 | TAX1BP1 | 0.871 | 2.550 | 0.342 | 0.012768083 | 0.800 | 0.666 | 0.934 |
| 68418 | cg03380643 | 20 | INSM1 | 1.520 | 3.105 | 0.490 | 0.025851718 | 0.799 | 0.665 | 0.933 |
| 429031 | cg24441627 | 12 | BRI3BP | 1.359 | 3.145 | 0.432 | 0.010672341 | 0.797 | 0.662 | 0.932 |
| 346203 | cg19142026 | 7 | HOXA4 | 4.162 | 14.063 | 0.296 | 3.48602E−25 | 0.797 | 0.662 | 0.932 |
| 128730 | cg06604058 | 11 | RTN3 | 4.502 | 9.796 | 0.460 | 1.51657E−09 | 0.797 | 0.662 | 0.932 |
| 395660 | cg22363327 | 6 | SFRS13B | 5.300 | 10.736 | 0.494 | 2.58184E−09 | 0.797 | 0.662 | 0.932 |
| 219099 | cg11688874 | 10 | WAC | 2.918 | 6.767 | 0.431 | 9.11319E−07 | 0.797 | 0.662 | 0.932 |
| 389248 | cg21914984 | 2 | CDC42EP3 | 1.929 | 4.295 | 0.449 | 0.00111894 | 0.795 | 0.660 | 0.930 |
| 355678 | cg19737664 | 11 | LRRC56 | 3.141 | 6.787 | 0.463 | 4.21674E−06 | 0.795 | 0.660 | 0.930 |
| 480467 | cg27552081 | 17 | WSB1 | 2.002 | 4.035 | 0.496 | 0.005458038 | 0.795 | 0.660 | 0.930 |
| 327760 | cg18003214 | 7 | GBX1 | 1.025 | 3.657 | 0.280 | 0.000108002 | 0.793 | 0.657 | 0.929 |
| 231390 | cg12425861 | 14 | PACS2 | 11.410 | 23.978 | 0.476 | 1.25951E−23 | 0.793 | 0.657 | 0.929 |
| 105622 | cg05310071 | 17 | PIGL | 1.343 | 2.822 | 0.476 | 0.035407019 | 0.793 | 0.657 | 0.929 |
| 75444 | cg03733219 | 19 | SPRED3 | 2.628 | 6.364 | 0.413 | 1.18731E−06 | 0.793 | 0.657 | 0.929 |
| 93392 | cg04672538 | 17 | ARSG; | 1.694 | 3.945 | 0.429 | 0.001622802 | 0.791 | 0.654 | 0.927 |
| | | | SLC16A6 | | | | | | | |
| 283564 | cg15313956 | 14 | CCDC88C | 24.615 | 53.012 | 0.464 | 1.40468E−27 | 0.791 | 0.654 | 0.927 |
| 25774 | cg01228134 | 2 | ECEL1 | 3.695 | 7.827 | 0.472 | 5.24938E−07 | 0.791 | 0.654 | 0.927 |
| 224036 | cg11964823 | 6 | MICB | 4.756 | 10.561 | 0.450 | 9.07618E−11 | 0.791 | 0.654 | 0.927 |
| 171657 | cg08894153 | 19 | ZNF709 | 3.697 | 7.690 | 0.481 | 1.18249E−06 | 0.789 | 0.652 | 0.926 |
| 212007 | cg11245569 | 11 | TRIM66 | 19.201 | 44.111 | 0.435 | 2.49994E−28 | 0.787 | 0.649 | 0.924 |
| 172735 | cg08957484 | 5 | CCNI2 | 2.006 | 4.026 | 0.498 | 0.005791874 | 0.785 | 0.646 | 0.923 |
| 376588 | cg21088281 | 4 | GPM6A | 2.276 | 4.861 | 0.468 | 0.000512335 | 0.785 | 0.646 | 0.923 |
| 218068 | cg11630226 | 8 | LY6K | 10.260 | 20.958 | 0.490 | 2.00438E−19 | 0.785 | 0.646 | 0.923 |
| 234984 | cg12637942 | 11 | NEAT1 | 2.068 | 4.257 | 0.486 | 0.002863149 | 0.785 | 0.646 | 0.923 |
| 178277 | cg09282338 | 20 | NXT1 | 1.956 | 4.687 | 0.417 | 0.000176068 | 0.785 | 0.646 | 0.923 |
| 227188 | cg12150111 | 6 | PPP1R3G | 2.437 | 5.071 | 0.481 | 0.000461752 | 0.785 | 0.646 | 0.923 |
| 296439 | cg16104283 | 1 | SDC3 | 1.822 | 4.038 | 0.451 | 0.002122225 | 0.785 | 0.646 | 0.923 |
| 231657 | cg12441052 | 11 | ZDHHC24; | 3.356 | 7.742 | 0.434 | 6.51988E−08 | 0.785 | 0.646 | 0.923 |
| | | | ACTN3 | | | | | | | |
| 445149 | cg25432323 | 16 | AARS | 1.522 | 3.190 | 0.477 | 0.018832674 | 0.783 | 0.644 | 0.921 |
| 211157 | cg11200917 | 5 | GLRA1 | 2.098 | 4.604 | 0.456 | 0.000647678 | 0.783 | 0.644 | 0.921 |
| 275000 | cg14781281 | 6 | HLA-J | 2.003 | 4.260 | 0.470 | 0.001998023 | 0.783 | 0.644 | 0.921 |
| 311010 | cg16943151 | 10 | RHOBTB1 | 20.464 | 45.644 | 0.448 | 2.86813E−28 | 0.783 | 0.644 | 0.921 |
| 481135 | cg27588119 | 17 | RNFT1 | 1.358 | 2.835 | 0.479 | 0.035794841 | 0.783 | 0.644 | 0.921 |
| 344453 | cg19021197 | 17 | TBX2 | 2.504 | 5.042 | 0.497 | 0.0007795 | 0.783 | 0.644 | 0.921 |
| 154316 | cg07936541 | 2 | ANKRD36B | 2.756 | 5.594 | 0.493 | 0.0002212 | 0.781 | 0.641 | 0.920 |
| 31482 | cg01519350 | 3 | ARMC8 | 2.925 | 6.312 | 0.463 | 1.40215E−05 | 0.781 | 0.641 | 0.920 |
| 92526 | cg04621255 | 9 | ENDOG | 3.028 | 6.074 | 0.498 | 9.90264E−05 | 0.781 | 0.641 | 0.920 |
| 90444 | cg04514249 | 4 | FREM3 | 2.102 | 5.199 | 0.404 | 2.66269E−05 | 0.781 | 0.641 | 0.920 |
| 247446 | cg13428516 | 19 | MAMSTR; | 5.751 | 12.030 | 0.478 | 3.04739E−11 | 0.781 | 0.641 | 0.920 |
| | | | RASIP1 | | | | | | | |
| 275466 | cg14807365 | 17 | SLC5A10; | 2.333 | 4.697 | 0.497 | 0.001550622 | 0.781 | 0.641 | 0.920 |
| | | | FAM83G | | | | | | | |
| 84708 | cg04217140 | 17 | ARRB2 | 1.797 | 3.649 | 0.493 | 0.010384289 | 0.778 | 0.639 | 0.918 |
| 124139 | cg06346696 | 3 | TUSC2 | 1.852 | 4.128 | 0.449 | 0.001632749 | 0.778 | 0.639 | 0.918 |
| 171006 | cg08862778 | 1 | MTOR | 3.085 | 6.231 | 0.495 | 6.23997E−05 | 0.778 | 0.639 | 0.918 |
| 462631 | cg26515694 | 19 | ZNF100 | 6.693 | 13.935 | 0.480 | 4.24012E−13 | 0.778 | 0.639 | 0.918 |
| 28019 | cg01346114 | 17 | GPS2 | 1.266 | 3.146 | 0.402 | 0.006704384 | 0.776 | 0.636 | 0.917 |
| 453286 | cg25969878 | 10 | STK32C | 8.709 | 18.328 | 0.475 | 6.62975E−18 | 0.776 | 0.636 | 0.917 |
| 360816 | cg20039944 | 12 | TRIAP1; GATC | 1.124 | 2.585 | 0.435 | 0.034563067 | 0.776 | 0.636 | 0.917 |
| 264059 | cg14219599 | 6 | GNL1; PRR3 | 1.512 | 3.393 | 0.446 | 0.007816111 | 0.774 | 0.633 | 0.915 |
| 258359 | cg13951491 | 1 | HPDL | 5.175 | 11.888 | 0.435 | 5.04143E−13 | 0.774 | 0.633 | 0.915 |
| 188227 | cg09858777 | 16 | NUDT16L1 | 1.653 | 3.795 | 0.436 | 0.002646575 | 0.774 | 0.633 | 0.915 |
| 5569 | cg00259755 | 10 | PWWP2B | 5.346 | 10.790 | 0.495 | 2.6544E−09 | 0.774 | 0.633 | 0.915 |
| 27937 | cg01341170 | 16 | SHISA9 | 1.250 | 2.679 | 0.467 | 0.040898446 | 0.774 | 0.633 | 0.915 |
| 441569 | cg25204764 | 1 | SRRM1 | 22.549 | 45.549 | 0.495 | 9.31694E−29 | 0.774 | 0.633 | 0.915 |
| 86955 | cg04330371 | 15 | NR2F2 | 4.541 | 9.507 | 0.478 | 1.27724E−08 | 0.772 | 0.631 | 0.914 |
| 92758 | cg04636402 | 5 | NRG2 | 5.246 | 11.315 | 0.464 | 4.34824E−11 | 0.772 | 0.631 | 0.914 |
| 351552 | cg19496491 | 11 | TEAD1 | 3.540 | 7.442 | 0.476 | 1.62304E−06 | 0.772 | 0.631 | 0.914 |
| 52515 | cg02579136 | 11 | WNT11 | 1.630 | 3.823 | 0.426 | 0.002042231 | 0.772 | 0.631 | 0.914 |
| 7342 | cg00347643 | 7 | YWHAG | 1.861 | 3.823 | 0.487 | 0.006787892 | 0.771 | 0.630 | 0.913 |
| 41246 | cg02010894 | 19 | CHERP | 1.376 | 3.139 | 0.438 | 0.011910573 | 0.770 | 0.628 | 0.912 |
| 100923 | cg05060949 | 7 | MNX1 | 3.555 | 9.204 | 0.386 | 1.89733E−11 | 0.770 | 0.628 | 0.912 |
| 74628 | cg03694515 | 18 | ZNF271; ZNF397OS | 1.666 | 3.501 | 0.476 | 0.010357084 | 0.770 | 0.628 | 0.912 |
| 306676 | cg16678169 | 2 | ALS2CR4 | 8.408 | 23.473 | 0.358 | 1.08748E−30 | 0.768 | 0.626 | 0.911 |
| 164947 | cg08522087 | 5 | ANKH | 2.516 | 5.681 | 0.443 | 2.98655E−05 | 0.768 | 0.626 | 0.911 |
| 180008 | cg09379601 | 19 | DNASE2 | 3.121 | 6.972 | 0.448 | 1.22613E−06 | 0.768 | 0.626 | 0.911 |
| 365547 | cg20358834 | 11 | LRFN4; PC | 1.161 | 2.787 | 0.416 | 0.018567368 | 0.768 | 0.626 | 0.911 |
| 410420 | cg23279021 | 5 | TMEM232 | 8.432 | 17.118 | 0.493 | 1.57839E−15 | 0.768 | 0.626 | 0.911 |
| 57273 | cg02816003 | 6 | RFX6 | 1.437 | 2.922 | 0.492 | 0.036082529 | 0.767 | 0.624 | 0.910 |
| 138366 | cg07082452 | 8 | EGR3 | 7.204 | 15.177 | 0.475 | 1.10105E−14 | 0.766 | 0.623 | 0.909 |
| 438908 | cg25030018 | 4 | STATH | 8.519 | 21.482 | 0.397 | 1.67867E−28 | 0.766 | 0.623 | 0.909 |
| 401498 | cg22753607 | 9 | ZCCHC7 | 1.370 | 2.988 | 0.458 | 0.02120068 | 0.766 | 0.623 | 0.909 |
| 122615 | cg06248741 | 2 | TXNDC9; EIF5B | 2.070 | 4.423 | 0.468 | 0.001338375 | 0.765 | 0.622 | 0.908 |
| 438512 | cg25010788 | 1 | NKAIN1 | 7.186 | 14.393 | 0.499 | 1.4341E−12 | 0.764 | 0.621 | 0.907 |
| 57757 | cg02841941 | 3 | P2RY1 | 2.294 | 4.856 | 0.472 | 0.000581404 | 0.764 | 0.621 | 0.907 |
| 357834 | cg19859486 | 3 | SACM1L | 2.313 | 4.667 | 0.496 | 0.001603348 | 0.764 | 0.621 | 0.907 |
| 244590 | cg13269439 | 11 | SF3B2 | 1.738 | 3.502 | 0.496 | 0.014311141 | 0.764 | 0.621 | 0.907 |
| 200318 | cg10543501 | 5 | HAND1 | 3.318 | 7.429 | 0.447 | 3.3809E−07 | 0.762 | 0.618 | 0.906 |
| 137824 | cg07055616 | 10 | NKX6-2 | 1.574 | 3.297 | 0.477 | 0.015530295 | 0.762 | 0.618 | 0.906 |
| 317667 | cg17351385 | 19 | ALKBH6 | 1.498 | 3.067 | 0.488 | 0.027164426 | 0.760 | 0.615 | 0.904 |
| 178850 | cg09315468 | 8 | DDHD2 | 1.645 | 4.369 | 0.377 | 0.000130863 | 0.760 | 0.615 | 0.904 |
| 398762 | cg22577136 | 1 | IKBKE | 1.297 | 2.732 | 0.475 | 0.040657983 | 0.760 | 0.615 | 0.904 |
| 282642 | cg15243856 | 20 | RBPJL; MATN4 | 5.997 | 12.089 | 0.496 | 1.56841E−10 | 0.760 | 0.615 | 0.904 |
| 165033 | cg08526825 | 16 | SRRM2 | 1.427 | 3.245 | 0.440 | 0.009702125 | 0.758 | 0.613 | 0.903 |
| 246686 | cg13390975 | 5 | BRIX1; RAD1 | 4.861 | 9.913 | 0.490 | 1.27944E−08 | 0.758 | 0.613 | 0.903 |
| 468705 | cg26862691 | 16 | CDK10 | 1.599 | 3.438 | 0.465 | 0.00980992 | 0.758 | 0.613 | 0.903 |
| 377175 | cg21126573 | 17 | KDM6B | 1.238 | 3.034 | 0.408 | 0.009555171 | 0.758 | 0.613 | 0.903 |
| 71380 | cg03531853 | 9 | KIF27 | 4.966 | 12.861 | 0.386 | 5.90555E−17 | 0.758 | 0.613 | 0.903 |
| 402800 | cg22831315 | 13 | SPG20 | 1.514 | 3.089 | 0.490 | 0.026782404 | 0.758 | 0.613 | 0.903 |
| 91524 | cg04569364 | 19 | ZNF17 | 1.584 | 3.494 | 0.453 | 0.007188554 | 0.758 | 0.613 | 0.903 |
| 414135 | cg23514016 | 5 | BHMT | 2.572 | 5.200 | 0.495 | 0.000534056 | 0.756 | 0.610 | 0.901 |
| 161164 | cg08304084 | 16 | SALL1 | 24.751 | 51.208 | 0.483 | 5.44043E−28 | 0.756 | 0.610 | 0.901 |
| 262955 | cg14172283 | 9 | TOMM5 | 1.058 | 2.424 | 0.436 | 0.047482595 | 0.756 | 0.610 | 0.901 |
| 473627 | cg27143049 | 11 | PDE3B; PSMA1 | 3.288 | 7.493 | 0.439 | 1.79972E−07 | 0.754 | 0.608 | 0.899 |
| 261572 | cg14102128 | 2 | SEPT10; | 1.454 | 2.973 | 0.489 | 0.031980288 | 0.754 | 0.608 | 0.899 |
| | | | ANKRD57 | | | | | | | |
| 398358 | cg22546168 | 10 | VENTX | 1.715 | 4.142 | 0.414 | 0.000689146 | 0.754 | 0.608 | 0.899 |
| 154968 | cg07973095 | 16 | DECR2 | 4.822 | 10.979 | 0.439 | 9.96539E−12 | 0.752 | 0.605 | 0.898 |
| 378163 | cg21181453 | 9 | DPM2 | 14.795 | 29.738 | 0.498 | 3.52766E−27 | 0.752 | 0.605 | 0.898 |
| 416548 | cg23664459 | 14 | INSM2 | 1.788 | 5.812 | 0.308 | 4.07583E−08 | 0.752 | 0.605 | 0.898 |
| 149132 | cg07650554 | 16 | SEPHS2 | 1.739 | 3.776 | 0.461 | 0.004528779 | 0.752 | 0.605 | 0.898 |
| 96541 | cg04840494 | 5 | SERINC5 | 1.231 | 2.697 | 0.456 | 0.035415669 | 0.752 | 0.605 | 0.898 |
| 238032 | cg12838902 | 7 | SLC29A4 | 4.466 | 9.446 | 0.473 | 1.02575E−08 | 0.752 | 0.605 | 0.898 |
| 350628 | cg19436567 | 6 | ARID1B | 1.753 | 3.665 | 0.478 | 0.007859256 | 0.749 | 0.603 | 0.896 |
| 392954 | cg22167789 | 19 | ONECUT3 | 2.917 | 6.280 | 0.465 | 1.59004E−05 | 0.749 | 0.603 | 0.896 |
| 26402 | cg01261044 | 14 | SRP54 | 1.510 | 3.117 | 0.485 | 0.023717941 | 0.749 | 0.603 | 0.896 |
| 402077 | cg22793735 | 3 | PLOD2 | 1.197 | 2.590 | 0.462 | 0.045528264 | 0.748 | 0.601 | 0.895 |
| 166947 | cg08634464 | 19 | ZNF57 | 11.731 | 5.679 | 2.066 | 3.20534E−12 | 0.747 | 0.600 | 0.895 |
| 484044 | ch.2.4639917R | 2 | ARMC9 | 1.198 | 2.865 | 0.418 | 0.016079026 | 0.745 | 0.598 | 0.893 |

The CpG methylation differences between CP and controls was ≥10% in all CpG targets suggesting a biological significance. That means that this level of methylation difference in a gene is likely to correlate with differences in actual gene transcription levels. Moreover, one microRNA (MIR-1469) was identified; and found to be linked with CP. Pathway and network analyses identified significant biological processes and functions related to these differentially methylated 262 genes, including: Axonal guidance and Actin cytoskeleton signaling, Wnt-signaling, Insulin receptor and PI3K/AKT signaling, TGF-B signaling, Crosstalk between Dendritic Cells and Natural Killer Cells, Neuroinflammation Signaling Pathway, Ephrin Receptor Signaling, Neuregulin Signaling and Tight Junction Signaling. Some of the critical genes identified and involved in the brain function are ADAM12, FGF8, PTEN, PDE3B, SMAD1, RUNX3 as well as miR-1469. This established that there is known biological significance of some of the genes that were found to be dysregulated in the analysis.

Validation by pyrosequencing. It was confirmed that the methylation state inferred by the Illumina HumanMethylation450K arrays data was not biased but represented true changes. The top 25 genes were selected for independent validation by pyrosequencing, based on their % methylation, AUC ROC, top fold change and EDR p-values. These analyses revealed similar methylation data as those calculated from the Illumina HumanMethylation450K arrays for all 25 genes. Bisulfite-converted genomic DNA was examined by quantitative pyrosequencing analysis. Detailed methodology was published previously.⁴⁹

Discussion. The present case control-based DNA methylation analysis was performed to explore the possible effect of gene methylation variation on the phenotype of subjects with cerebral palsy. Wth these results, possible pathway mechanisms linked to genes differentially methylated in this disorder were investigated. In this study, numerous hypomethylated markers were identified in genes in cerebral palsy patients that were significantly different from control subjects. Among, a total of 4 CpG loci (cg01561596, cg03586379, cg08052428 and cg07898899) in 4 genes individually had excellent predictive accuracy (AUC≥0.90) for the detection of CP. Additionally, a good predictive accuracy for CP detection was achieved at 120 CpG biomarkers accuracy (AUC≥0.80). The methylation markers were found to be covering coding genes, miRNA, small nucleolar RNAs and non-coding RNAs. Among the genes identified in the study, a total of 69 genes were under the influence of 10 canonical pathway mechanisms identified using the IPA tool. The major canonical pathways with significant relationship with brain function along with few important genes are discussed further.

Axonal guidance and Actin cytoskeleton signaling. Axonal guidance is mainly mediated by Wnt proteins. In cerebral cortex, the Wnt-signaling regulates the migrating neurons. Neuronal migration disruption is involved in several neurodevelopment disorders including cerebral palsy. Wnt proteins binds to the Frizzled transmembrane receptor to activate G proteins, which increase intracellular calcium levels. Intracellular calcium level disruption is one of the causes of bone fragility. In children with cerebral palsy, disruption in bone homeostasis results in microdamage that in turn predisposes children to non-traumatic fractures. Wnt proteins also have a major role in inducing Rho-dependent changes in the actin cytoskeleton. Wingless-Type Mmtv Integration Site Family, Member 11 (WNT11) (OMIM 603699) on chromosome 11q13.5, which belongs to Wnt family of proteins, and ADAM12 (OMIM 602714) on chromosome 10q26.2) are hypo-methylated in our study. ADAM12 has a major role in reorganizing the actin cytoskeleton during early adipocyte differentiation. Impairment of the actin cytoskeleton contributes to neuromotor damage, a pathogenic mechanism in cerebral palsy. Fibroblast Growth Factor 8 (FGF8) (OMIM 600483) on chromosome 10q24.32 was another hypo-methylated gene, which has implications during early embryogenesis. The null mutation of this gene in mice confers lethality at an early embryonic stage with malformation of major brain structures. This implies the importance of normal level expression of these genes, and a potential patho-mechanism of differential methylation leading to CP in our study population.

Insulin receptor and PI3K/AKT signaling. Impairment in serine/threonine phosphorylation of insulin receptor substrate proteins leads to insulin resistance, which could have pathophysiological implications in CP. Phosphorylation impairment decreases binding of the downstream enzyme PI3K, altering the activation of kinase Akt. Akt upregulation is a response to ischemia and reperfusion, while ischemia is one of the major causes associated with CP. Interruptions in the interlinked insulin and PI3K/Akt signaling pathways may lead to fatal effects in case of CP. Phosphatase and tensin homolog (PTEN) (OMIM 601728) on chromosome 10q23.31 is one of the differentially methylated gene under PI3K/Akt influence and has been identified as candidate tumor suppressor gene as well as an important molecule for brain growth. It regulates brain growth by interacting with Ctnnb1 and with β-catenin signaling. PTEN plays role in neuronal development and survival, synaptic plasticity and axonal regeneration and been linked with neurodegenerative disorders. PDE3B (OMIM 60204) on chromosome 11p15.2 which is under the insulin receptor signaling mechanism, combines with JAK2/PI3K pathways to play a neuroprotective role in the presence of G-CSF factor. Thus, the disruption of these complex interaction implicates a potential causative role CP.

TGF-β signaling. Muscle contracture is one of the common clinical states in CP. The contracture in cerebral palsy induces changes in types of muscle collagen via transforming growth factor β (TGF-β). TGF-β signaling also plays a significant role in several neurodegenerative disorders as it normally has neuroprotective properties and initiates protection against excitotoxicity. Neuronal TGF-β, which has a role in tissue regeneration, cell differentiation, and regulation of the immune system, interacts with IL-9 with effects such as the development of periventricular leukomalacia, a major cause of cerebral palsy. SMAD proteins are intracellular signaling molecules for the TGF-β family, bone morphogenic protein (BMP) family, growth, and differentiation factor (GDF) family, Müllerian inhibitory factors (MIS), activins and inhibins. SMAD1 (OMIM 601595) on chromosome 4q31.21 has a role in neuronal development, differentiation and dedifferentiation and Runt-Related Transcription Factor 3 (RUNX3) (OMIM 600210) on chromosome 1p36.11, has a crucial role in cranial sensory neuron development. These two genes were found to be hypo-methylated in the present study, and are known to be involved in anomalous neuronal development might have contributed to CP in our subjects.

miR-1469 in CP. MicroRNAs (miRNAs) are important in cell developmental processes like proliferation, differentiation, cell cycling and apoptosis. Along with these processes, miRNAs were also observed to be involved in neural cell patterning, establishment, neuronal plasticity, and neurogenesis. One of the miRNAs, miR-1469, was identified to be differentially methylated in our study with a p-value of 1.27724E-08. Differential expression of this marker has already been observed to be associated with neurological complications including glioblastoma multiforme, amyotrophic lateral sclerosis, temporal lobe epilepsy and DiGeorge syndrome. One study revealed that miR-1469 regulated multiple targets in Parkinson disease. In the present study, miR-1469 may have a crucial role in regulating the transcription process in CP manifestation. In conclusion, the panel of CpG methylation biomarkers identified in this study using genome-wide methylation analysis revealed many gene targets that possibly impacts pathogenic mechanisms such as non-traumatic fractures, neuromotor damage, ischemia, neuronal development, and survival damage. The responsible genes are under the influence of canonical pathways like Axonal guidance signaling, Actin cytoskeleton signaling, Insulin receptor signaling, PI3K/AKT signaling, TGF-B signaling, Neuregulin signaling, Ephrin receptor signaling, Crosstalk between Dendritic cells and Natural killer cells, and Tight junction signaling. miR-1469 has also been identified in brain-associated disorders with a possible mechanism yet to be identified. The genes identified hold significant potential as biomarkers for early detection of prenatal or antenatal damage prior to the appearance of clinical symptoms of CP. Further, they could potentially be targets for novel therapeutic interventions for CP.

SUPPLEMENTARY TABLE S1A
MicroRNA (miRNA)

| | | | | % Methylation | % Methylation | Fold | | | | |
|---|---|---|---|---|---|---|---|---|---|---|
| Index | TargetID | CHR | Gene | Cases | Control | change | FDR p-Val | AUC | CI_lower | CI_upper |
| 86955 | cg04330371 | 15 | miR1469 | 4.540631 | 9.506502 | 0.477634255 | 1.27724E−08 | 0.772256729 | 0.630843034 | 0.913670423 |

SUPPLEMENTARY TABLE S1B
Open reading Frames (ORF)

| | | | | % Methylation | % Methylation | | | | | |
|---|---|---|---|---|---|---|---|---|---|---|
| Index | TargetID | CHR | Gene | Cases | Control | Fold chance | FDR p-Val | AUC | CI_lower | CI_upper |
| 243288 | cg13187827 | 6 | C6orf27 | 12.87842 | 27.46615 | 0.468883335 | 4.56185E−28 | 0.937888199 | 0.860827886 | 1 |
| 442956 | cg25302370 | 6 | C6orf165 | 1.553326 | 3.110247 | 0.499422072 | 0.029072697 | 0.819875776 | 0.691808583 | 0.94794297 |
| 400744 | cg22704520 | 2 | C2orf47; | 5.018259 | 10.16143 | 0.493853621 | 9.52142E−09 | 0.80952381 | 0.678296024 | 0.940751595 |
| | | | C2orf60 | | | | | | | |
| 161571 | cg08326511 | 2 | C2orf76 | 1.398478 | 2.923954 | 0.478283174 | 0.03057326 | 0.801242236 | 0.667594073 | 0.934890399 |
| 390824 | cg22028544 | 8 | C8orf59 | 0.8438922 | 2.2806 | 0.370030781 | 0.033580702 | 0.797101449 | 0.662277878 | 0.931925021 |
| 224540 | cg11995490 | 7 | C7orf50 | 23.59414 | 47.79116 | 0.493692557 | 1.73565E−28 | 0.790890269 | 0.654345896 | 0.927434642 |
| 143000 | cg07318050 | 1 | C1orf57 | 2.160747 | 4.538459 | 0.476097063 | 0.001276677 | 0.786749482 | 0.649085558 | 0.924413407 |
| 291269 | cg15790941 | 4 | C4orf34 | 1.755345 | 3.51999 | 0.498678974 | 0.014432288 | 0.786749482 | 0.649085558 | 0.924413407 |
| 314696 | cg17173767 | 8 | C8orf84 | 1.957124 | 4.614223 | 0.424150285 | 0.000261211 | 0.786749482 | 0.649085558 | 0.924413407 |
| 113295 | cg05733554 | 14 | C14orf37 | 1.386784 | 3.473194 | 0.399282044 | 0.002824463 | 0.775362319 | 0.634730482 | 0.915994155 |
| 262751 | cg14162940 | 20 | C20orf160 | 4.411848 | 9.393991 | 0.469645755 | 9.26983E−09 | 0.772256729 | 0.630843034 | 0.913670423 |
| 368491 | cg20556702 | 21 | C21orf91 | 5.308687 | 11.92654 | 0.445115432 | 1.30435E−12 | 0.751552795 | 0.605216793 | 0.897888797 |

SUPPLEMENTARY TABLE S1C
SNOR

| | | | | % | | | | | | |
|---|---|---|---|---|---|---|---|---|---|---|
| | | | | Methylation | % Methylation | | | | | |
| Index | TargetID | CHR | Gene | Cases | Control | Fold chance | FDR p-Val | AUC | CI_lower | CI_upper |
| 304543 | cg16565409 | 17 | SNORD4A | 15.66457 | 36.19498 | 0.432782944 | 2.48296E−29 | 0.805383023 | 0.672933311 | 0.937832734 |

SUPPLEMENTARY TABLE S1D
NCRNA

| | | | | % | % | | | | | |
|---|---|---|---|---|---|---|---|---|---|---|
| | | | | Methylation | Methylation | | | | | |
| Index | TargetID | CHR | Gene | Cases | Control | Fold chance | FDR p-Val | AUC | CI_lower | CI_upper |
| 275000 | cg14781281 | 6 | NCRNA00171 | 2.003294 | 4.26048 | 0.470203827 | 0.001998023 | 0.782608696 | 0.643846916 | 0.921370476 |
| 388139 | cg21846177 | 20 | NCRNA00028 | 4.017215 | 11.38221 | 0.35293805 | 1.83373E−16 | 0.805383023 | 0.672933311 | 0.937832734 |

SUPPLEMENTARY TABLE S1E
LOC

| | | | | % | | | | | | |
|---|---|---|---|---|---|---|---|---|---|---|
| | | | | Meth- | % | | | | | |
| | | | | ylation | Methylation | | | | | |
| Index | TargetID | CHR | Gene | Cases | Control | Fold chance | FDR p-Val | AUC | CI_lower | CI_upper |
| 219695 | cg11722376 | 2 | LOC389033 | 7.813488 | 16.61209 | 0.470349486 | 1.88544E−16 | 0.830227743 | 0.705478733 | 0.954976754 |
| 195068 | cg10241347 | 10 | LOC399815 | 5.783334 | 13.59514 | 0.425397164 | 1.0669E−15 | 0.811594203 | 0.680986326 | 0.94220208 |
| 16644 | cg00788028 | 2 | LOC440839 | 6.232712 | 13.17966 | 0.472903853 | 1.09491E−12 | 0.797101449 | 0.662277878 | 0.931925021 |
| 352953 | cg19580633 | 5 | LOC100268168 | 1.480319 | 3.563958 | 0.41535815 | 0.003063357 | 0.801242236 | 0.667594073 | 0.934890399 |
| 165033 | cg08526825 | 16 | LOC100128788 | 1.426822 | 3.245075 | 0.439688451 | 0.009702125 | 0.757763975 | 0.612852693 | 0.902675257 |

Summary. Blood spots were collected on filter paper from newborns undergoing routine screening for metabolic disorders. Newborns averaged 2 days of age at the time of collection. Completely de-identified (to lab researchers) residual blood spots not used for metabolic testing was stored at room temperature at the Michigan Department of Community Health facilities in Lansing, Mich. DNA was extracted and purified from a single spot of blood on filter paper as described previously in the application and methylation levels in different CPG islands determined using the Illumina's Infinium Human Methylation450 Bead Chip system as described earlier.

The level or percentage methylation at multiple cytosine throughout the DNA was compared in 23 cases of CP versus 21 normal cases. Table 1 shows 220 cytosine loci located in 220 known genes (i.e. intragenic) that were associated with significant differences in methylation between CP cases and the normal cases. Threshold FDR p-value<0.05 and AUC 0.75 were used. The GENE ID number(s) and GENE symbols, chromosome number on which the gene is located, position of the cytosine locus displaying differential methylation and DNA strand (reverse or forward) are provided along with the contribution (marginal contribution) of each particular cytosine locus for the overall prediction of CP versus unaffected cases. The low False Discovery Rate (FDR) values, high fold change in methylation of cases relative to controls and high AUROC (AUC) curve values taken together indicate the highly significant differences in the percentage methylation between these specific cytosines in CP cases versus controls and the diagnostic utility of the methylation level at these molecular sites for the detection of CP.

### EXAMPLE 2

In the same analysis of bloodspots from the patients previously described in EXAMPLE 1 we focused on the extragenic cytosines (Table 2). The level or percentage methylation at multiple (extragenic) cytosine loci throughout the DNA was compared in CP versus unaffected controls. Table 2 shows 76 cytosine loci located external to known genes that were associated with significant differences in methylation between CP cases and unaffected controls. Although these loci are extragenic, extragenic loci are known to interact with genes that are located distant from the sequences, designated as ‘interacting genes” in the tables. The low False Discovery Rate (FDR) values, high fold change in methylation level of cases relative to controls and high AUROC curve values in combination indicate the highly significant differences in the methylation levels between these specific cytosines in CP cases versus unaffected controls and the diagnostic utility of the methylation level at these molecular sites for the detection of CP.

TABLE 2
Extragenic CpG sites

| | | | | | | Log FC | | | | | |
|---|---|---|---|---|---|---|---|---|---|---|---|
| | | | | | Fold | LOG | % Methylation | % Methylation | | | |
| Index | TargetID | CHR | LOG10p | FDR p-Val | chance | log2 (FC) | Cases | Control | AUC | CI_lower | CI_upper |
| 455336 | cg26099834 | 15 | −29.04 | 9.12587E−30 | 0.35 | −0.46 | 9.94 | 28.67 | 0.93 | 0.84 | 1.00 |
| 56741 | cg02785814 | 11 | −5.65 | 2.21863E−06 | 0.48 | −0.32 | 3.58 | 7.44 | 0.92 | 0.83 | 1.00 |
| 245054 | cg13298199 | 1 | −7.74 | 1.82372E−08 | 0.49 | −0.31 | 4.82 | 9.81 | 0.91 | 0.82 | 1.00 |
| 107560 | cg05406088 | 15 | −29.70 | 2.00062E−30 | 0.30 | −0.53 | 6.82 | 22.91 | 0.90 | 0.80 | 1.00 |
| 331947 | cg18238374 | 14 | −6.96 | 1.09202E−07 | 0.32 | −0.49 | 1.85 | 5.75 | 0.90 | 0.80 | 1.00 |
| 86867 | cg04324666 | 19 | −6.12 | 7.65999E−07 | 0.50 | −0.31 | 4.08 | 8.24 | 0.87 | 0.76 | 0.98 |
| 432165 | cg24634568 | 1 | −19.46 | 3.4722E−20 | 0.38 | −0.42 | 5.60 | 14.75 | 0.87 | 0.76 | 0.98 |
| 303631 | cg16519487 | 13 | −7.67 | 2.1417E−08 | 0.40 | −0.40 | 3.00 | 7.46 | 0.87 | 0.76 | 0.98 |
| 412418 | cg23404528 | 2 | −8.58 | 2.65027E−09 | 0.45 | −0.34 | 4.27 | 9.41 | 0.87 | 0.76 | 0.98 |
| 166127 | cg08587775 | 19 | −19.57 | 2.68345E−20 | 0.48 | −0.32 | 10.03 | 20.95 | 0.86 | 0.75 | 0.98 |
| 352749 | cg19567689 | 14 | −16.84 | 1.43701E−17 | 0.48 | −0.32 | 8.90 | 18.46 | 0.86 | 0.74 | 0.97 |
| 14767 | cg00698771 | 1 | −21.02 | 9.51341E−22 | 0.33 | −0.48 | 4.52 | 13.64 | 0.85 | 0.73 | 0.97 |
| 64123 | cg03156443 | 6 | −4.13 | 7.42365E−05 | 0.45 | −0.35 | 2.45 | 5.44 | 0.84 | 0.72 | 0.96 |
| 409916 | cg23250574 | 6 | −8.74 | 1.81914E−09 | 0.49 | −0.31 | 5.33 | 10.83 | 0.84 | 0.72 | 0.96 |
| 139688 | cg07146104 | 1 | −1.60 | 0.024978782 | 0.49 | −0.31 | 1.52 | 3.12 | 0.84 | 0.72 | 0.96 |
| 292769 | cg15881107 | 5 | −21.55 | 2.84847E−22 | 0.46 | −0.34 | 9.62 | 21.06 | 0.84 | 0.72 | 0.96 |
| 389005 | cg21901277 | 2 | −3.12 | 0.000761672 | 0.44 | −0.36 | 1.93 | 4.37 | 0.84 | 0.72 | 0.96 |
| 279 | cg00011740 | 16 | −2.22 | 0.005957388 | 0.44 | −0.36 | 1.50 | 3.44 | 0.84 | 0.72 | 0.96 |
| 281634 | cg15174791 | 10 | −27.12 | 7.65714E−28 | 0.49 | −0.31 | 26.50 | 53.65 | 0.83 | 0.71 | 0.96 |
| 377132 | cg21123519 | 14 | −30.22 | 6.00427E−31 | 0.37 | −0.43 | 8.28 | 22.37 | 0.83 | 0.71 | 0.96 |
| 482494 | ch.1.183610071R | 1 | −3.07 | 0.000857472 | 0.36 | −0.44 | 1.33 | 3.64 | 0.83 | 0.70 | 0.95 |
| 127780 | cg06548479 | 8 | −27.80 | 1.58448E−28 | 0.47 | −0.33 | 21.03 | 45.05 | 0.83 | 0.70 | 0.95 |
| 366483 | cg20422417 | 2 | −29.42 | 3.7638E−30 | 0.47 | −0.33 | 15.24 | 32.41 | 0.83 | 0.70 | 0.95 |
| 473324 | cg27125849 | 17 | −2.18 | 0.006636357 | 0.45 | −0.35 | 1.58 | 3.51 | 0.83 | 0.70 | 0.95 |
| 193507 | cg10157715 | 17 | −5.38 | 4.19031E−06 | 0.43 | −0.37 | 2.68 | 6.22 | 0.82 | 0.69 | 0.95 |
| 434511 | cg24766821 | 2 | −2.91 | 0.00122115 | 0.41 | −0.39 | 1.59 | 3.88 | 0.82 | 0.69 | 0.95 |
| 141406 | cg07227769 | 11 | −17.44 | 3.67085E−18 | 0.48 | −0.32 | 9.08 | 18.92 | 0.82 | 0.69 | 0.95 |
| 220763 | cg11786255 | 5 | −12.86 | 1.37082E−13 | 0.28 | −0.55 | 2.31 | 8.16 | 0.82 | 0.69 | 0.95 |
| 194977 | cg10236452 | 1 | −10.82 | 1.51363E−11 | 0.30 | −0.52 | 2.25 | 7.49 | 0.82 | 0.69 | 0.95 |
| 302834 | cg16472050 | 2 | −2.55 | 0.0028149 | 0.50 | −0.30 | 2.21 | 4.43 | 0.82 | 0.69 | 0.95 |
| 408556 | cg23178550 | 7 | −14.66 | 2.16436E−15 | 0.49 | −0.31 | 8.48 | 17.13 | 0.82 | 0.69 | 0.95 |
| 239585 | cg12940965 | 4 | 8.65 | 2.21985E−09 | 2.22 | 0.35 | 8.58 | 3.86 | 0.81 | 0.68 | 0.94 |
| 380619 | cg21336435 | 12 | −12.27 | 5.35235E−13 | 0.49 | −0.31 | 7.02 | 14.34 | 0.81 | 0.68 | 0.94 |
| 381832 | cg21433231 | 17 | −6.29 | 5.09144E−07 | 0.40 | −0.40 | 2.60 | 6.46 | 0.81 | 0.68 | 0.94 |
| 266945 | cg14362630 | 9 | −1.35 | 0.045125525 | 0.49 | −0.31 | 1.35 | 2.76 | 0.81 | 0.68 | 0.94 |
| 282913 | cg15261861 | 12 | −7.54 | 2.86113E−08 | 0.46 | −0.34 | 4.02 | 8.71 | 0.81 | 0.68 | 0.94 |
| 399599 | cg22634378 | 19 | −7.33 | 4.68223E−08 | 0.50 | −0.30 | 4.71 | 9.51 | 0.81 | 0.68 | 0.94 |
| 451349 | cg25835226 | 10 | −10.98 | 1.04529E−11 | 0.37 | −0.43 | 3.31 | 8.95 | 0.81 | 0.68 | 0.94 |
| 10545 | cg00497232 | 4 | −7.86 | 1.38658E−08 | 0.49 | −0.31 | 4.74 | 9.75 | 0.81 | 0.68 | 0.94 |
| 294103 | cg15965134 | 3 | −4.16 | 6.94425E−05 | 0.49 | −0.31 | 3.05 | 6.17 | 0.81 | 0.68 | 0.94 |
| 319471 | cg17464350 | 17 | −2.44 | 0.003598108 | 0.37 | −0.43 | 1.16 | 3.16 | 0.81 | 0.68 | 0.94 |
| 187859 | cg09838568 | 21 | −7.21 | 6.22646E−08 | 0.49 | −0.31 | 4.61 | 9.33 | 0.80 | 0.67 | 0.94 |
| 363440 | cg20218280 | 7 | −8.25 | 5.62366E−09 | 0.48 | −0.32 | 4.69 | 9.82 | 0.80 | 0.67 | 0.94 |
| 54863 | cg02695467 | 19 | −1.93 | 0.011706248 | 0.42 | −0.37 | 1.29 | 3.04 | 0.80 | 0.67 | 0.93 |
| 457051 | cg26193372 | 2 | −4.20 | 6.2405E−05 | 0.39 | −0.41 | 1.84 | 4.73 | 0.80 | 0.67 | 0.93 |
| 27868 | cg01337391 | 16 | −2.26 | 0.005541666 | 0.42 | −0.38 | 1.41 | 3.36 | 0.80 | 0.66 | 0.93 |
| 369102 | cg20596329 | 11 | −2.25 | 0.005644734 | 0.47 | −0.33 | 1.77 | 3.76 | 0.80 | 0.66 | 0.93 |
| 355017 | cg19704288 | 4 | 7.37 | 4.2773E−08 | 2.03 | 0.31 | 8.71 | 4.29 | 0.79 | 0.66 | 0.93 |
| 485558 | rs6426327 | | −24.76 | 1.74413E−25 | 0.40 | −0.40 | 25.67 | 64.92 | 0.79 | 0.66 | 0.93 |
| 233916 | cg12580752 | 3 | −3.12 | 0.000760474 | 0.41 | −0.39 | 1.63 | 4.03 | 0.79 | 0.65 | 0.92 |
| 420249 | cg23906459 | 8 | −1.73 | 0.018543391 | 0.49 | −0.31 | 1.60 | 3.28 | 0.79 | 0.65 | 0.92 |
| 96896 | cg04856590 | 6 | −1.54 | 0.028676855 | 0.47 | −0.33 | 1.35 | 2.89 | 0.79 | 0.65 | 0.92 |
| 84827 | cg04222358 | 3 | −6.75 | 1.77496E−07 | 0.40 | −0.40 | 2.72 | 6.78 | 0.78 | 0.65 | 0.92 |
| 452028 | cg25888561 | 10 | −10.48 | 3.28714E−11 | 0.43 | −0.37 | 4.35 | 10.18 | 0.78 | 0.64 | 0.92 |
| 199730 | cg10513943 | 5 | −26.78 | 1.6729E−27 | 0.47 | −0.33 | 25.62 | 54.38 | 0.78 | 0.64 | 0.92 |
| 72792 | cg03599078 | 10 | −1.96 | 0.010865348 | 0.48 | −0.32 | 1.71 | 3.54 | 0.78 | 0.64 | 0.92 |
| 258350 | cg13951074 | 9 | −2.22 | 0.006071049 | 0.48 | −0.32 | 1.86 | 3.85 | 0.78 | 0.64 | 0.92 |
| 70829 | cg03506502 | 4 | −9.57 | 2.69742E−10 | 0.49 | −0.31 | 5.68 | 11.59 | 0.77 | 0.63 | 0.92 |
| 128508 | cg06590268 | 5 | −1.72 | 0.019117845 | 0.48 | −0.32 | 1.56 | 3.22 | 0.77 | 0.63 | 0.92 |
| 380596 | cg21334513 | 6 | −17.45 | 3.50862E−18 | 0.45 | −0.34 | 7.77 | 17.14 | 0.77 | 0.63 | 0.91 |
| 242311 | cg13125506 | 9 | −29.59 | 2.54723E−30 | 0.42 | −0.37 | 12.04 | 28.54 | 0.77 | 0.63 | 0.91 |
| 448047 | cg25617012 | 4 | −3.94 | 0.000115924 | 0.48 | −0.32 | 2.78 | 5.75 | 0.77 | 0.62 | 0.91 |
| 62465 | cg03066081 | 17 | −5.59 | 2.57889E−06 | 0.49 | −0.31 | 3.63 | 7.48 | 0.76 | 0.62 | 0.91 |
| 365608 | cg20362689 | 8 | −27.14 | 7.28027E−28 | 0.49 | −0.31 | 26.36 | 53.41 | 0.76 | 0.62 | 0.91 |
| 484551 | ch.4.2941683R | 4 | −8.90 | 1.26813E−09 | 0.49 | −0.31 | 5.49 | 11.10 | 0.76 | 0.62 | 0.91 |
| 16528 | cg00782260 | 1 | −2.83 | 0.001463473 | 0.46 | −0.34 | 1.97 | 4.29 | 0.76 | 0.62 | 0.91 |
| 370633 | cg20691507 | 6 | −5.33 | 4.63832E−06 | 0.50 | −0.30 | 3.72 | 7.47 | 0.76 | 0.62 | 0.91 |
| 131455 | cg06743703 | 13 | −10.52 | 2.98949E−11 | 0.44 | −0.36 | 4.67 | 10.62 | 0.76 | 0.61 | 0.90 |
| 157360 | cg08108965 | 1 | −21.31 | 4.92226E−22 | 0.49 | −0.31 | 11.97 | 24.18 | 0.76 | 0.61 | 0.90 |
| 343545 | cg18959044 | 2 | −3.57 | 0.00026819 | 0.48 | −0.32 | 2.53 | 5.29 | 0.75 | 0.61 | 0.90 |
| 184453 | cg09636849 | 2 | −1.42 | 0.038140095 | 0.42 | −0.37 | 1.05 | 2.48 | 0.75 | 0.60 | 0.90 |
| 95091 | cg04765857 | 16 | −28.82 | 1.51937E−29 | 0.49 | −0.31 | 19.24 | 38.88 | 0.75 | 0.60 | 0.89 |
| 128836 | cg06610548 | 17 | −6.93 | 1.16379E−07 | 0.50 | −0.30 | 4.52 | 9.11 | 0.75 | 0.60 | 0.89 |
| 482821 | ch.10.295680R | 10 | −1.73 | 0.018436474 | 0.39 | −0.41 | 1.03 | 2.65 | 0.75 | 0.60 | 0.89 |
| 150381 | cg07719621 | 16 | −1.90 | 0.012695589 | 0.49 | −0.31 | 1.73 | 3.52 | 0.74 | 0.60 | 0.89 |
| 216603 | cg11538389 | 1 | −4.73 | 1.87947E−05 | 0.43 | −0.36 | 2.48 | 5.72 | 0.74 | 0.59 | 0.89 |

### EXAMPLE 3

Diagnostic Accuracy of Methylation Markers and Demographic characteristics for CP Detection. Only limited demographic information was available from patient birth certificates and provided by the Michigan Department of Community Health (MDCH). Based on the terms of the Internal Review Board (IRB). The demographic features were newborn gender, birth weight, gestational age at delivery, maternal age, interval between birth and sample collection (in hours), and time in years between specimen collection and molecular analysis. These and other demographic and clinical factors can be combined with cytosine methylation data using statistical techniques previously described-logistic regression, evolutionary computing etc. to develop further predictive algorithms and to estimate CP risk.

### EXAMPLE 4

Diagnostic Accuracy of Methylation Markers for Detection of Overall CP Group Based on Logistic Regression Analysis. As previously noted, logistic regression analysis can be used to estimate individual risk of CP and based on this sensitivity and specificity values calculated. Because of the small number of overall CP cases used herein, there was insufficient study power to calculate sensitivity and specificity values for individual sub-categories of CP. As a result, this particular analysis was limited to the overall (combined) CP group versus normal. Logistic regression analysis was performed using the “R” computer program (version 3.2.2.). A combination of CpG loci (in separate genes were used to calculate sensitivity and specificity values.

The top 8 CpG sites for predicting, detecting, and/or diagnosing CP are cg12425861, cg19499452, cg08894153, cg24455365, cg13187827, cg12204727, cg03586379, and cg08634464.

The logistic regression analysis for the combination of 8 CpG sites: Best model achieved AUC=1, Sens=100%, Spec=100%, and Accuracy=100% by using eight CpG (selected by mSVM-RFE).

### Logistic Regression /using Artificial Intelligence and Deep Learning

Data Preprocessing. No missing values were detected in the data sets. To adjust for the offset between high and low-intensity features, and to reduce the heteroscedasticity, the log value of each methylation value centered by its mean (x̅) and auto scaled by its standard deviation (s). Quantile normalization is used to reduce sample-to-sample variation.

Deep Learning (DL). Generally classical machine learning techniques make predictions directly from a set of features that have been pre-specified by the user. However, representation learning techniques transform features into some intermediate representation prior to mapping them to final predictions. Deep Learning (DL) is a form of representation learning that uses multiple transformation steps to create very complex features. DL is widely applied in pattern recognition, image processing, computer vision, and recently in bioinformatics. DL is categorized into feed-forward artificial neural networks (ANNs), which uses more than one hidden layer (y) that connects the input (x) and output layer (z) via a weight (VV) matrix. The weight matrix W which is expected to minimize the difference between the input layer (x) and the output layer (z) is considered as the best one and chosen by the system to get the best results.

Machine Learning Algorithms. A representative set of five machine learning classification algorithms which have been applied for problems of data classification in metabolomics and genomics studies can be selected and the results of these five machine learning algorithms compared with deep learning. Random forest (RF) is a widely used machine learning algorithm based on decision tree theory. It works with high-dimensional data and can deal with unbalanced and missing values in the data. Support vector machine (SVM) is another machine learning algorithm that separates the metabolomics data with N data points into (N-1) dimensional hyperplane. SVM has the advantage of avoiding over-fitting and uses the kernel trick for more complex problems to get better results by changing the kernel function. Generalized Linear Model (GLM) measures the relationship between the categorical dependent variable and one or more independent variables by estimating probabilities using a logistic function, which is the cumulative logistic distribution. The output of a GLM is more informative than other classification algorithms. Prediction Analysis for Microarrays (PAM) is a statistical technique for class prediction from gene expression data using nearest shrunken centroids. This method identifies the subsets of genes that best characterize each class and gives satisfying results in metabolomics and genomics studies as well. Linear Discriminant Analysis (LDA) is closely related to analysis of variance (ANOVA) and regression analysis, which also attempt to express one dependent variable as a linear combination of other features or measurements.

Software Packages Utilized. The H2O R package (https://cran.r-project.org/web/packages/h2o/h2o.pdf, Author The H2O.ai team Maintainer Tom Kraljevic <tomk@0xdata.com>) was used to tune the parameters of the DL model.

To get the optimal predictions for the artificial intelligence algorithms other than DL, the caret R package (https://cran.r-project.org/web/packages/caret/caret.pdf, Maintainer Max Kuhn <mxkuhn@gmail.com>) was used to tune the parameters in the models.

The variable importance functions varimp in h2o and varImp in caret R packages were used to rank the models features in each of the predictive algorithms.

The pROC R package was used to compute area under the curve (AUC) of a receiver-operating characteristic (ROC) curve to assess the overall performance of the models.

Modeling & Evaluation. The data are split into 80% training set and 20% testing set. While dealing with a small and medium size of data in the machine learning applications, the 80/20 split is a commonly used one. A 10-fold cross validation was performed on the 80% training data during the model construction process, and the model was tested on the hold out 20% of data. To avoid sampling bias, the above splitting process was repeated ten times and calculated the average AUC on the 10 hold out test sets. In addition to AUC, sensitivity, specificity, and 95% confidence intervals for the test sets were calculated.

The following parameters were used to tune the DL model and other machine learning algorithms: for DL model Epochs (number of passes of the full training set), I1 (penalty to converge the weights of the model to 0), I2 (penalty to prevent the enlargement of the weights), input dropout ratio (ratio of ignored neurons in the input layer during training), andnumber of hidden layers; for SVM model, cost of classification; for RF model, number of trees to fit; and for PAM model, threshold amount for shrinking toward the centroid.

One of the problems in DL model is its overfitting complications. To avoid overfitting in the DL model, three regularization parameters were used. L1, which increases model stability and causes many weights to become 0 and L2, which prevents weights enlargement. L1 lets only strong weights survive (constant pulling force towards zero), while L2 prevents any single weight from getting too big. Dropout has recently been introduced as a powerful generalization technique, and is available as a parameter per layer, including the input layer. The key idea is to randomly drop units (along with their connections) from the neural network during training. This prevents units from co-adapting too much. The third parameter used for avoiding overfitting in DL model is input_dropout_ratio which controls the amount of input layer neurons that are randomly dropped (set to zero), controls overfitting with respect to the input data (useful for high-dimensional noisy data).

Feature Importance. Feature (predictor) importance is estimated using a model-based approach. In other words, a feature is considered important if it contributes to the predictive model performance. Variable importance functions varimp in h2o and varImp in caret R packages were used to rank the models features in each of the predictive algorithms.

Results. The primary data set (in this case 220 epigenomic biomarkers) can be divided up into 5 -6 equal number of CpG loci or subgroups and analyzed separately. Then each subgroup is evaluated separately (epigenomic biomarker only) and also combined with the clinical and demographic predictors or risk factors for CP for evaluation. Next, all the epigenomic biomarkers of the primary data set in one group are analyzed and the performance differences are observed. The second subgroup as one group is then analyzed to see the performance results of epigenomic markers with and without clinical and demographic markers. For every group, the top epigenomic markers or epigenomic and clinical markers are analyzed and ranked.

The aim is to assess the predictive ability of the DL framework to separate CP patients using genomics data. Toward this goal, preprocessing steps (log transformation, centering, autoscaling, and quantile normalization) are applied before constructing the DL model. Before training the model, the model is pre-trained using autoencoder and the whole data without labels. This step improves the model performance, avoids random initialization of the weights, and selects the best model architecture. Subsequently, the DL model is trained using a wide range of parameters (as stated in Modeling & Evaluation section) and selected the best model with the minimum mean square error.

DL is subsequently compared with five other commonly used artificial intelligence methods: RF, SVM, LDA, PAM, and GLM, bearing in mind the strengths of the different approaches. The average AUCs, sensitivity and specificity values calculated on the hold out (validation) test sets are then reported. Higher area under the ROC curve value is often achieved with DL than other AI methods. In addition, higher sensitivity and specificity values are often achieved with DL than other AI methods, too.

The subject matter described above is provided by way of illustration only and should not be construed as limiting. Various modifications and changes may be made to the subject matter described herein without following the example embodiments and applications illustrated and described, and without departing from the true spirit and scope of the present invention, which is set forth in the following claims.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

### REFERENCES

1. Bax M, Goldstein M, Rosenbaum P, Leviton A, Paneth N, Dan B, et al. Proposed definition and classification of cerebral palsy, April 2005. Dev Med Child Neurol. 2005;47(8):571-6.
2. The Definition and Classification of Cerebral Palsy. Dev Med Child Neurol. 2007;49(s109):1-44.
3. Benda W, McGibbon NH, Grant KL: Improvements in muscle symmetry in children with cerebral palsy after equine-assisted therapy (hippotherapy). J Altem Complement Med 2003, 9(6):817-825.
4. Lundy C, Lumsden D, Fairhurst C: Treating complex movement disorders in children with cerebral palsy. Ulster Med J 2009, 78(3):157-163.
5. Moreno-De-Luca A, Ledbetter DH, Martin CL: Genetic [corrected] insights into the causes and classification of [corrected] cerebral palsies. Lancet Neurol 2012, 11(3):283-292.
6. Bottcher L: Children with spastic cerebral palsy, their cognitive functioning, and social participation: a review. Child Neuropsychol 2010, 16(3):209-228.
7. Colver A, Fairhurst C, Pharoah P O: Cerebral palsy. Lancet 2014, 383(9924):1240-1249.
8. Romeo D M, Sini F, Brogna C, Albamonte E, Ricci D, Mercuri E: Sex differences in cerebral palsy on neuromotor outcome: a critical review. Dev Med Child Neurol 2016, 58(8):809-813.
9. Wu Y W, Xing G, Fuentes-Afflick E, Danielson B, Smith L H, Gilbert W M: Racial, ethnic, and socioeconomic disparities in the prevalence of cerebral palsy. Pediatrics 2011, 127(3):e674-681.
10. Van Naarden Braun K, Doernberg N, Schieve L, Christensen D, Goodman A, Yeargin-Allsopp M: Birth Prevalence of Cerebral Palsy: A Population-Based Study. Pediatrics 2016, 137(1).
11. Shamsoddini A, Amirsalari S, Hollisaz M T, Rahimnia A, Khatibi-Aghda A: Management of spasticity in children with cerebral palsy. Iran J Pediatr 2014, 24(4):345-351.
12 .Knezevic-Pogancev M: [Cerebral palsy and epilepsy]. Med Pregl 2010, 63(7-8):527-530.
13. Zwaigenbaum L: The intriguing relationship between cerebral palsy and autism. Dev Med Child Neurol 2014, 56(1):7-8.
14. MacLennan A H, Thompson S C, Gecz J: Cerebral palsy: causes, pathways, and the role of genetic variants. Am J Obstet Gynecol 2015, 213(6):779-788.
15. Nelson K B, Dambrosia J M, lovannisci D M , Cheng S, Grether J K, Lammer E: Genetic polymorphisms and cerebral palsy in very preterm infants. Pediatr Res 2005, 57(4):494-499.
16. Khankhanian P, Baranzini S E, Johnson B A, Madireddy L, Nickles D, Croen L A, Wu Y W: Sequencing of the 1L6 gene in a case-control study of cerebral palsy in children. BMC Med Genet 2013, 14:126.
17. Lerer I, Sagi M, Meiner V, Cohen T, Zlotogora J, Abeliovich D: Deletion of the ANKRD15 gene at 9p24.3 causes parent-of-origin-dependent inheritance of familial cerebral palsy. Hum Mol Genet 2005, 14(24):3911-3920.
18. McMichael G, Girirajan S, Moreno-De-Luca A, Gecz J, Shard C, Nguyen L S, Nicholl J, Gibson C, Haan E, Eichler E et al: Rare copy number variation in cerebral palsy. Eur J Hum Genet 2014, 22(1):40-45.
19. Oskoui M, Gazzellone M J, Thiruvahindrapuram B, Zarrei M, Andersen J, Wei J, Wang Z, Wntle R F, Marshall C R, Cohn R D et al: Clinically relevant copy number variations detected in cerebral palsy. Nat Commun 2015, 6:7949.
20. McMichael G, Bainbridge M N, Haan E, Corbett M, Gardner A, Thompson S, van Bon B W, van Eyk C L, Broadbent J, Reynolds C et al: Whole-exome sequencing points to considerable genetic heterogeneity of cerebral palsy. Mol Psychiatry 2015, 20(2):176-182.
21. Schoendorfer N C, Obeid R, Moxon-Lester L, Sharp N, Vitetta L, Boyd R N, Davies P S: Methylation capacity in children with severe cerebral palsy. Eur J Clin Invest 2012, 42(7):768-776.
22. Bundey S, Griffiths M I. Recurrence risks in families of children with symmetrical spasticity. Developmental medicine and child neurology. 1977;19(2):179-91.
23. Hemminki K, Sundquist K, Li X. Familial risks for main neurological diseases in siblings based on hospitalizations in Sweden. Twin research and human genetics : the official journal of the International Society for Twin Studies. 2006;9(4):580-6.
24. Lynex C N, Carr I M, Leek J P, Achuthan R, Mitchell S, Maher E R, et al. Homozygosity for a missense mutation in the 67 kDa isoform of glutamate decarboxylase in a family with autosomal recessive spastic cerebral palsy: parallels with Stiff-Person Syndrome and other movement disorders. BMC neurology. 2004;4(1):20.
25. Lerer I, Sagi M, Meiner V, Cohen T, Zlotogora J, Abeliovich D. Deletion of the ANKRD15 gene at 9p24.3 causes parent-of-origin-dependent inheritance of familial cerebral palsy. Human molecular genetics. 2005;14(24):3911-20.
26. Petterson B, Stanley F, Henderson D. Cerebral palsy in multiple births in Western Australia: genetic aspects. American journal of medical genetics. 1990;37(3):346-51.
27. Fletcher N A, Foley J. Parental age, genetic mutation, and cerebral palsy. Journal of medical genetics. 1993;30(1):44-6.
28. Kuroda M M, Weck M E, Sarwark J F, Hamidullah A, Wainwright M S. Association of apolipoprotein E genotype and cerebral palsy in children. Pediatrics. 2007;119(2):306-13.
29. Gibson C S, MacLennan A H, Hague W M, Haan E A, Priest K, Chan A, et al. Associations between inherited thrombophilias, gestational age, and cerebral palsy. American journal of obstetrics and gynecology. 2005;193(4):1437.
30. O'Callaghan M E, Maclennan A H, Gibson C S, McMichael G L, Haan E A, Broadbent J L, et al. Fetal and maternal candidate single nucleotide polymorphism associations with cerebral palsy: a case-control study. Pediatrics. 2012;129(2):e414-23.
31. Gibson C S, MacLennan A H, Goldwater P N, Haan E A, Priest K, Dekker G A, et al. The association between inherited cytokine polymorphisms and cerebral palsy. American journal of obstetrics and gynecology. 2006;194(3):674 el-11.
32. Gibson C S, Maclennan A H, Dekker G A, Goldwater P N, Sullivan T R, Munroe D J, et al. Candidate genes and cerebral palsy: a population-based study. Pediatrics. 2008;122(5):1079-85.
33. Ozanne S E, Constancia M. Mechanisms of disease: the developmental origins of disease and the role of the epigenotype. Nature clinical practice Endocrinology & metabolism. 2007;3(7):539-46.
34. Fleiss B, Gressens P. Tertiary mechanisms of brain damage: a new hope for treatment of cerebral palsy? Lancet neurology. 2012;11(6):556-66.
35. Favrais G, van de Looij Y, Fleiss B, Ramanantsoa N, Bonnin P, Stoltenburg-Didinger G, et al. Systemic inflammation disrupts the developmental program of white matter. Annals of neurology. 2011;70(4):550-65.
36. (Fatemi M et al. Footprints of mammalian CpG DNA methyltransferases revealing nucleosome positions at a single molecule level. Nucleic Acids Res 2005; 33:e176)
37. (Hanley J A, McNeil B J. Radiology 1982; 143:29-36)
38. (Ziong and Laird, Nucleic Acid Res 1997 25; 2532-4
39. (Eads et al, Cancer Res 1999; 59:2302-2306)
40. (Gonzalgo and Jones Nuclei Acids Res1997; 25:252-31)
41. (Eckhart F, Lewin J, Cortese R et al: DNA methylation profiling of human chromosome 6, 20 and 22. Nat Gent. 38, 1379-85. 2006)
42. (Royston P, Thompson S G. Model-based screening by risk with application in Down's syndrome. Stat Med 1992;11:257-68.)
43. (Wald N J, Cuckle H S, Deusem J W et al (1988) Maternal serum screening for down syndrome in early pregnancy. BMJ 297, 883-887.)
44. [Penza-Reyes C A, Sipper M. Evolutionary computation in medicine 2000;19:1-23
45. Artif Intell Med 2000;19:1-23
46. Whitley D. An overview of evolutionary algorithms: practical issues and common pitfalls. Info Software Tech 2001;43:87-31].
47. [Goodcare R. Making sense of the metabolome using evolutionary computing: seeing the wood with the trees. J Exp Bot 2005;56:245-54.]
48. Miranda V, Srinivasan D, Proenca LM. Evolutionary computation in power systems. Elec Power Energ Sys 1998;20:89-981
49. Radhakrishna U, Albayrak S, Alpay-Savasan Z, Zeb A, Turkoglu O, Sobolewski P, Bahado-Singh R O: Genome-Wde DNA Methylation Analysis and Epigenetic Variations Associated with Congenital Aortic Valve Stenosis (AVS). PLoS One 2016, 11(5):e0154010.
50. Onishi K, Hollis E, Zou Y: Axon guidance and injury-lessons from Wnts and Wnt signaling. Curr Opin Neurobiol 2014, 27:232-240.
51. Boitard M, Bocchi R, Egervari K, Petrenko V, Viale B, Gremaud S, Zgraggen E, Salmon P, Kiss J Z: Wnt signaling regulates multipolar-to-bipolar transition of migrating neurons in the cerebral cortex. Cell Rep 2015, 10(8):1349-1361.
52. Tsutsui Y, Nagahama M, Mizutani A: Neuronal migration disorders in cerebral palsy. Neuropathology 1999, 19(1):14-27.
53. Houlihan C M , Stevenson R D: Bone density in cerebral palsy. Phys Med Rehabil Clin N Am 2009, 20(3):493-508.
54. Fontaine R, Mesples B, Lelievre V, Gressens P: 125 TGF-Beta-1 Mediates IL-9/Mast Cells Interactions in a Mouse Model of Periventricular Leukomalacia. Pediatric Research 2005, 58(2):376.
55. Kawaguchi N, Sundberg C, Kveiborg M, Moghadaszadeh B, Asmar M, Dietrich N, Thodeti C K, Nielsen F C, Moller P, Mercurio A M et al: ADAM12 induces actin cytoskeleton and extracellular matrix reorganization during early adipocyte differentiation by regulating betal integrin function. J Cell Sci 2003, 116(Pt 19):3893-3904.
56. Kruer M C, Jepperson T, Dutta S, Steiner R D, Cottenie E, Sanford L, Merkens M, Russman B S, Blasco P A, Fan G et al: Mutations in gamma adducin are associated with inherited cerebral palsy. Ann Neurol 2013, 74(6):805-814.
57. Sunmonu N A, Li K, Li J Y: Numerous isoforms of Fgf8 reflect its multiple roles in the developing brain. J Cell Physiol 2011, 226(7):1722-1726.
58. Peterson M D, Gordon P M, Hurvitz E A, Burant C F: Secondary muscle pathology and metabolic dysregulation in adults with cerebral palsy. Am J Physiol Endocrinol Metab 2012, 303(9):E1085-1093.
59. Rask-Madsen C, Kahn C R: Tissue-specific insulin signaling, metabolic syndrome, and cardiovascular disease. Arterioscler Thromb Vasc Biol 2012, 32(9):2052-2059.
60. Mullonkal C J, Toledo-Pereyra L H: Akt in ischemia and reperfusion. J Invest Surg 2007, 20(3):195-203.
61. Babcock M A, Kostova F V, Ferriero D M, Johnston M V, Brunstrom J E, Hagberg H, Maria B L: Injury to the preterm brain and cerebral palsy: clinical aspects, molecular mechanisms, unanswered questions, and future research directions. J Child Neurol 2009, 24(9):1064-1084.
62. Chen Y, Huang W-C, Séjourné J, Clipperton-Allen A E, Page D T: <em>Pten</em> Mutations Alter Brain Growth Trajectory and Allocation of Cell Types through Elevated β-Catenin Signaling. The Journal of Neuroscience 2015, 35(28):10252-10267.
63. Ismail A, Ning K, Al-Hayani A, Sharrack B, Azzouz M: PTEN: a molecular target for neurodegenerative disorders. Translational Neuroscience 2012, 3(2):132-142.
64. Charles M S, Drunalini Perera P N, Doycheva D M, Tang J: Granulocyte-colony stimulating factor activates JAK2/PI3K/PDE3B pathway to inhibit corticosterone synthesis in a neonatal hypoxic-ischemic brain injury rat model. Exp Neurol 2015, 272:152-159.
65. Jung S T, Seo H Y, Lee J J, Kim M S, Kim Y K, Kim G J: Increased Expression of the TGF-Isoform and Changed Contents of Collagen in Tendon of Cerebral Palsy Patients. [symbol] 2004, 39(5):531-536.
66. Dobolyi A, Vincze C, Pal G, Lovas G: The neuroprotective functions of transforming growth factor beta proteins. Int J Mol Sci 2012, 13(7):8219-8258.
67. Kulak-Bejda A, Kulak P, Bejda G, Krajewska-Kulak E, Kulak W: Stem cells therapy in cerebral palsy: A systematic review. Brain Dev 2016, 38(8):699-705.
68. Chambers S M, Fasano C A, Papapetrou E P, Tomishima M, Sadelain M, Studer L: Highly efficient neural conversion of human ES and iPS cells by dual inhibition of SMAD signaling. Nat Biotechnol 2009, 27(3):275-280.
69. Park B Y, Saint-Jeannet J P: Expression analysis of Runx3 and other Runx family members during Xenopus development. Gene Expr Patterns 2010, 10(4-5):159-166.
70. Yoon B H, Jun J K, Romero R, Park K H, Gomez R, Choi J H, Kim I O: Amniotic fluid inflammatory cytokines (interleukin-6, interleukin-1beta, and tumor necrosis factor-alpha), neonatal brain white matter lesions, and cerebral palsy. Am J Obstet Gynecol 1997, 177(1):19-26.
71. Greenberg D S, Soreq H: MicroRNA therapeutics in neurological disease. Curr Pharm Des 2014, 20(38):6022-6027.
72. Wang W, Kwon E J, Tsai L H: MicroRNAs in learning, memory, and neurological diseases.

Learn Mem 2012, 19(9):359-368.
73. Rivera-Diaz M, Miranda-Roman M A, Soto D, Quintero-Aguilo M, Ortiz-Zuazaga H, Marcos-Martinez M J, Vivas-Mejia P E: MicroRNA-27a distinguishes glioblastoma multiforme from diffuse and anaplastic astrocytomas and has prognostic value. Am J Cancer Res 2015, 5(1):201-218.
74. Freischmidt A, Muller K, Zondler L, Weydt P, Volk A E, Bozic A L, Walter M, Bonin M, Mayer B, von Arnim C A et al: Serum microRNAs in patients with genetic amyotrophic lateral sclerosis and pre-manifest mutation carriers. Brain 2014, 137(Pt 11):2938-2950.
75. Kan A A, van Erp S, Derijck A A, de Wit M, Hessel E V, O′Duibhir E, de Jager W, Van Rijen P C, Gosselaar P H, de Graan P N et al: Genome-wide microRNA profiling of human temporal lobe epilepsy identifies modulators of the immune response. Cell Mol Life Sci 2012, 69(18):3127-3145.
76. de la Morena M T, Eitson J L, Dozmorov I M, Belkaya S, Hoover A R, Anguiano E, Pascual M V, van Oers N S: Signature MicroRNA expression patterns identified in humans with 22q11.2 deletion/DiGeorge syndrome. Clin Immunol 2013, 147(1):11-22.
77. Santosh P S, Arora N, Sarma P, Pal-Bhadra M, Bhadra U: Interaction map and selection of microRNA targets in Parkinson's disease-related genes. J Biomed Biotechnol 2009, 2009:363145.
78. Liu Y, Aryee M J, Padyukov L, Fallin M D, Hesselberg E, Runarsson A, Reinius L, Acevedo N, Taub M, Ronninger M et al: Epigenome-wide association data implicate DNA methylation as an intermediary of genetic risk in rheumatoid arthritis. Nat Biotechnol 2013, 31(2):142-147.
79. Zhang C, Wang L, Chen L, Ren W, Mei A, Chen X, Deng Y: Two novel mutations of the NCSTN gene in Chinese familial acne inverse. J Eur Acad Dermatol Venereol 2013, 27(12):1571-1574.
80. Wilhelm-Benartzi C S, Koestler D C, Karagas M R, Flanagan J M, Christensen B C, Kelsey K T, Marsit C J, Houseman E A, Brown R: Review of processing and analysis methods for DNA methylation array data. Br J Cancer 2013, 109(6):1394-1402.
81. Daca-Roszak P, Pfeifer A, Zebracka-Gala J, Rusinek D, Szybinska A, Jarzab B, Wtt M, Zietkiewicz E: Impact of SNPs on methylation readouts by Illumina Infinium HumanMethylation450 BeadChip Array: implications for comparative population studies. BMC Genomics 2015, 16(1):1003.
82. Gu. Z: ComplexHeatmap: Making Complex Heatmaps. R package version 1.6.0. https://qithubcom/jokergoo/ComplexHeatmap2015.
83. Huberman L, Boychuck Z, Shevell M et al. Age at referral of children for initial diagnosis of cerebral palsy and rehabilitation: Current practices. J Child Neurol. 2016; 31:364-9.
84. Hadders-Algra M. Early diagnosis and early intervention in cerebral palsy. Frontiers in Neurology. 2014; 5:1-13).
85. Bosanquet M, Copeland I, Ware R et al. A systematic review of tests to predict cerebral palsy in young children. Dev Med Child Neurol. 2013; 55:418-26.
Hadders-Algra M. Early diagnosis and early intervention in cerebral palsy. Frontiers in Neurology. 2014; 5:1-13.
Bosanquet M, Copeland I, Ware R et al. A systemetic review of tests to predict cerebral palsy in young children. Dev Med Child Neurol. 2013; 55:418-26.
86. Mirmiran M, Barnes P D, Keller K, et al. Neonatal brain magnetic resonance imaging before discharge is better than serial cranial ultrasound in predicting cerebral palsy in very low birth weight preterm infants. Pediatrics 2004;114: 992-8.
87. Vanderveen J A, Bassler D, Robertson C M et al. Early interventions involving parents to improve neurodevelopmental outcomes of premature infants: a meta-analysis. J Perinatol. 2009;29:342-51.
88. McCormick M C, Brooks-Gunn J, Burka S L et al. Early intervention in low birth weight premature infants: Results at 18 years of age for the infant health development program. Pediatrics. 2006; 117:771-80.
89. Noritz G H. “Screening, Listening to Parents Key to Early CP Diagnosis”. AAP News, Dec. 13, 2017, http://www.aappublications.org/news/2017/12/13/CerebralPalsyl21317.
90. Chatterjee R, Vinson C. Biochemica et Biophisica Acta 2012;1819: 763-70.
91. Davies M N, Volta M, Pidsley R et al. Functional annotation of human brain methylation identifies tissue-specific epigenetic variation across brain and blood. Genome Biol. 2012; 13:1-14.
92. Lui J, Chen J, Ehrilich S et al. Methylation patterns in whole blood correlate with symptooms in schizophrenia subjects. Schizophrenia Bulletin. 2014; 40:769-776.
93. Song Y, Miyaki K, Suzuki T et al. Altered DNA methylation status of human brain derived neutophils factor gene could be useful as biomarker of depression. Am J of Genet Part B.
2014; 9999:1-18.

### REFERENCES FOR ARTIFICIAL INTELLIGENCE

[1] Hinton, Geoffrey E., Nitish Srivastava, Alex Krizhevsky, Ilya Sutskever, and Ruslan R.

Salakhutdinov. “Improving neural networks by preventing co-adaptation of feature detectors.” arXiv preprint arXiv:1207.0580 (2012).
[2] Srivastava, Nitish, Geoffrey Hinton, Alex Krizhevsky, Ilya Sutskever, and Ruslan Salakhutdinov. “Dropout: a simple way to prevent neural networks from overfitting.” The Journal of Machine Learning Research 15, no. 1 (2014): 1929-1958.
[3] Pasa, Luca, and Alessandro Sperduti. “Pre-training of recurrent neural networks via linear autoencoders.” In Advances in Neural Information Processing Systems, pp. 3572-3580. 2014.
[4] Min, S., Lee, B., & Yoon, S. (2017). Deep learning in bioinformatics, Briefings in bioinformatics, 18(5), 851-869.
[5] Angermueller, C., Parnamaa, T., Parts, L., & Stegle, 0. (2016). Deep learning for computational biology. Molecular systems biology, 12(7), 878.
[6] \Mtten, I. H., Frank, E., Hall, M. A., & Pal, C. J. (2016). Data Mining: Practical machine learning tools and techniques. Morgan Kaufmann.
[7] Aiakwaa, F. M., Chaudhary, K., & Garmire, L. X. (2018). Deep learning accurately predicts estrogen receptor status in breast cancer metabolomics data. Journal of proteome research.

1. A method for predicting or diagnosing cerebral palsy (CP) in a patient, wherein the method comprises:
obtaining a sample from the patient;
extracting nucleic acid from the sample;
assaying the nucleic acid to determine a frequency or percentage methylation of cytosine at one or more genomic loci; and
comparing the cytosine methylation level of the patient to a control and/or to a CP patient group

2. The method of claim 1, wherein the method further comprises calculating the individual risk of CP based on the cytosine methylation level at different sites throughout the genome.

3. The method of claim 1, wherein the one or more loci comprise at least two genomic loci.

4. The method of claim 1, wherein the one or more loci are selected from Table 1.

5. The method of claim 1, wherein the one or more loci are selected from Table 1 and have an AUC of 0.75 or greater, 0.80 or greater, 0.85 or greater, 0.90 or greater, or 0.95 or greater.

6. The method of claim 1, wherein the one or more loci are selected from Table S1A, Table S1 B, Table S1C, Table S1 D, or Table S1E.

7. The method of claim 1, wherein the percentage methylation of cytosines are determined for different combinations of loci to calculate the probability of CP in the subject.

8. The method of claim 1, wherein the assay is a bisulfite-based methylation assay or a whole genome methylation assay.

9. The method of claim 1, wherein measurement of the frequency or percentage methylation of cytosine nucleotides is obtained using gene or whole genome sequencing techniques.

10. The method of claim 1, wherein the nucleic acid comprises DNA or RNA.

11. The method of claim 1, wherein the RNA comprises miRNA or mRNA

12. The method of claim 10, wherein the DNA is obtained from cells.

13. The method of claim 12, wherein the DNA comprises cell free DNA.

14. The method of claim 13, wherein the DNA is extracted from body fluid.

15. The method of claim 14, wherein the body fluid comprises blood, plasma, serum, urine, saliva, sputum, amniotic fluid, cervical fluid or secretion, urine, tear, sweat, placental tissue, or a buccal swab.

16. The method of claim 1, wherein the patient is an embryo, a fetus, a newborn, or a pediatric patient.

17. The method of any one of claims 1, further comprising determining the risk or predisposition to having a CP at any time during any period of postnatal life.

18. The method of claim 1, wherein the method further comprises treating the patient postnatally with therapy, medication, and/or surgery.

19. The method of claim 1, wherein the one or more loci comprise cg12425861, cg19499452, cg08894153, cg24455365, cg13187827, cg12204727, cg03586379, or cg08634464.

20. The method of claim 1, wherein the loci comprise cg12425861, cg19499452, cg08894153, cg24455365, cg13187827, cg12204727, cg03586379, and cg08634464.
